(19) United States
(12) Patent Application Publication
(10) Pub. No.: US 2024/0216573 A1
(43) Pub. Date: Jul. 4, 2024

(54) COMPOSITIONS COMPRISING CROSS-LINKED HYALURONIC ACID AND METHODS OF USE THEREOF

(71) Applicant: HALLURA LTD., Yokneam (IL)

(72) Inventors: Stephane MEUNIER, Munich (DE); Svetlana SHNEIDER, Haifa (IL); Libi CHIRUG, Givat Ela (IL); Tal EYAL, Beney Yehuda (IL)

(21) Appl. No.: 18/286,630

(22) PCT Filed: Apr. 13, 2022
(86) PCT No.: PCT/IL22/50391

§ 371 Date: Oct. 12, 2023

Related U.S. Application Data
(60) Provisional application No. 63/184,962, filed on May 6, 2021.
(60) Provisional application No. 63/174,111, filed on Apr. 13, 2021.

(51) Int. Cl. A61L 27/18; A61F 2/00; A61L 27/50

(52) U.S. Cl. CPC A61L 27/18; A61F 2/0059; A61L 27/50; A61L 2400/06

(57) ABSTRACT

The present invention is directed to a composition comprising a polymer comprising a first hyaluronic acid (HA) chain and a second HA chain crosslinked via a linker comprising an unsaturated moiety or a derivative thereof coupled to a tetrazine moiety or a derivative thereof, wherein a weight ratio between water soluble HA (sHA) and the polymer within the composition is less than 10%. In some embodiments, the polymer of the invention is characterized by having a crosslinking degree of 0.6 to 4%.

### CROSS REFERENCE TO RELATED APPLICATIONS

This application claims the benefit of priority from U.S. Provisional Patent Applications No. 63/174,111 filed on Apr. 13, 2021 entitled “COMPOSITIONS COMPRISING CROSS-LINKED HYALURONIC ACID, AND METHODS OF USE THEREOF; and from U.S. Provisional Patent Application No. 63/184,962 filed on May 6, 2021 entitled “COMPOSITIONS COMPRISING HYALURONIC ACID AND CROSS-LINKED HYALURONIC ACID, AND METHODS OF USE THEREOF”, the contents of which are incorporated herein by reference in their entirety.

### FIELD OF INVENTION

The present invention, in some embodiments thereof, relates to cross-linked hyaluronic acid.

### BACKGROUND OF THE INVENTION

Crosslinking of hyaluronic acid (HA) in dermal fillers confers the desired mechanical properties and in particular the lifting (or volumizing) properties of the material.

The desire to use a material as close as possible to the endogenous hyaluronic acid, dictates the production of material having low degrees of modification (e.g., % crosslinking). Both hyaluronic acid and cross-linked hyaluronic acids are known to be highly hygroscopic. Therefore, when these materials are injected into the skin tissue, they have tendency to attract water in the tissue and lead to swelling and edema. Water attraction positively correlates with the content of hyaluronic acid and cross-linked hyaluronic acid. Accordingly, due to the need to reduce the risk of swelling and edema it is desirable to produce dermal fillers with low content of hyaluronic acid and cross-linked HA.

Commercially available HA-based dermal fillers are known to comprise high content of water-soluble HA fractions (up to about 40% w/w of the total HA content). The water-soluble HA fractions (sHA) are usually composed of low to medium molecular weight HA chains, and/or HA oligosaccharides characterized by an average molecular weight (MW) of less than 700 kDa, specifically MW of less than 500 kDa.

Low-molecular-weight HA (e.g., MW of 10-300 kDa) were found to induce pro-inflammatory effects in various myeloid cells, including microglia, as confirmed by numerous studies. Furthermore, increased levels of low molecular weight (LMW) HA have been found in many lung disorders including asthma, pulmonary fibrosis, COPD, etc., as well as other inflammatory diseases like rheumatoid arthritis. Additionally, it has been demonstrated that LMW-HA can also induce angiogenesis and tumor progression. Accordingly, there is an unmet need to reduce the sHA content of the dermal fillers.

The addition of a fraction of unmodified HA to dermal fillers is a common practice. This unmodified HA is not inter-connected with the cross-linked matrix and can be released from the gel as a soluble fraction in water. It has the purpose to decrease the extrusion forces of the gel through fine needles by lubrication and to enhance hydration of the tissue surrounding the implantation sites. The typical addition of unmodified HA is done on the already cross-linked matrix and has the effect to break the gel homogeneity and to dramatically reduce the gel's mechanical properties. There is a need for development of compositions comprising the desired homogeneity and flowability without reduction of the mechanical properties, and methods for producing such compositions.

### SUMMARY OF THE INVENTION

The present invention, in some embodiments thereof, relates to a cross-linked hyaluronic acid.

In some embodiments, the one or more linkers is represented by

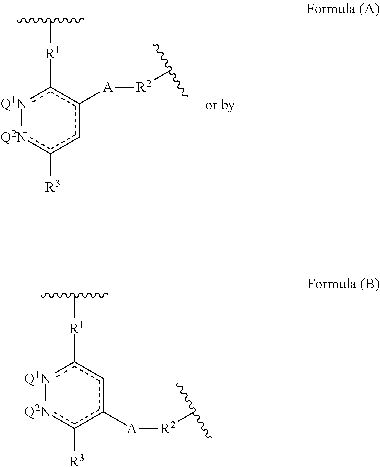

or a combination thereof; wherein: [symbol] represents a single or a double bond; R¹, R², or both, are selected from the group consisting of: a bond, alkyl, alkenyl, cycloalkyl, aryl, heteroaryl, heteroalicyclic, C(O)—NH-alkyl-NH, and alkyl-NZ wherein Z is a bond, aryl, or heteroaryl; A is selected form the group consisting of: a bond, alkyl, and aryl; Q¹, Q² or both represent hydrogen, or are absent; and wherein R³ is selected from the group consisting of: hydrogen, alkyl, aryl, or heteroaryl substituted or non-substituted.

In some embodiments, the unsaturated moiety comprises a styrene moiety or a derivative thereof and wherein the styrene moiety is covalently bound to the first HA chain or to the second HA chain.

In some embodiments, the styrene moiety or a derivative thereof is represented by formulae IA-D:

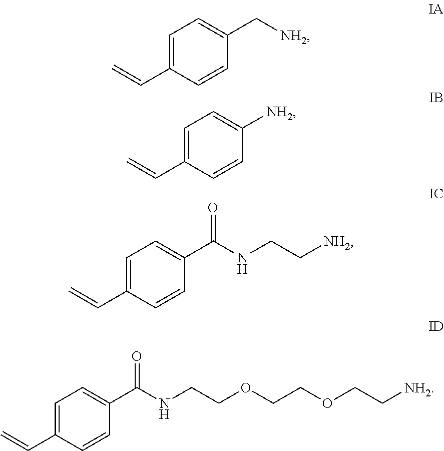

In some embodiments, the one or more linkers is represented by

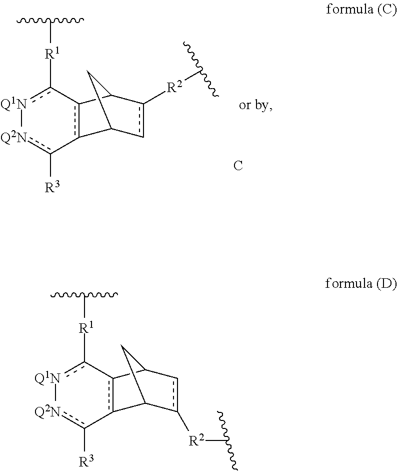

or a combination thereof; wherein: [symbol] represents a single or a double bond; R¹, R², or both, are selected from the group consisting of: a bond, alkyl, alkenyl, cycloalkyl, aryl, heteroaryl, heterocyclyl, C(O)—NH-alkyl-NH, and alkyl-NZ wherein Z is a bond, aryl, or heteroaryl; Q¹, Q² or both represent hydrogen, or are absent; and R³ is selected from the group consisting of: hydrogen, alkyl, aryl, or heteroaryl substituted or non-substituted.

In some embodiments, the one or more linkers is derived from:
(a) a norbornene moiety represented by any of Formulae IIA-G:

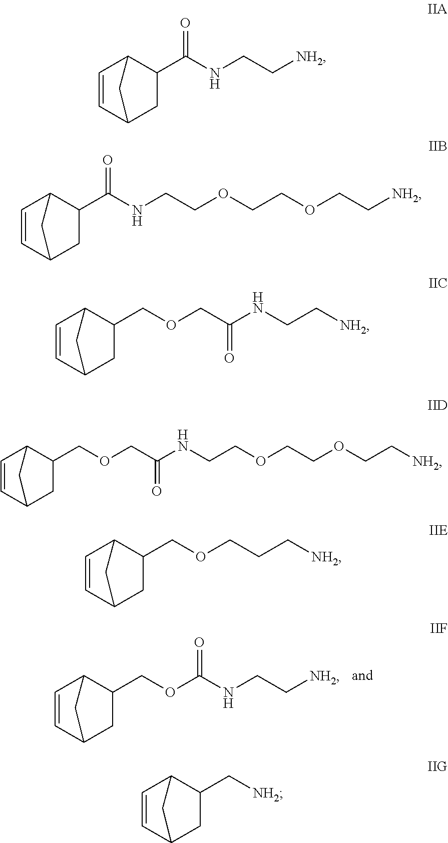

and from (b) a tetrazine moiety represented by any of formulae IIIA-F:

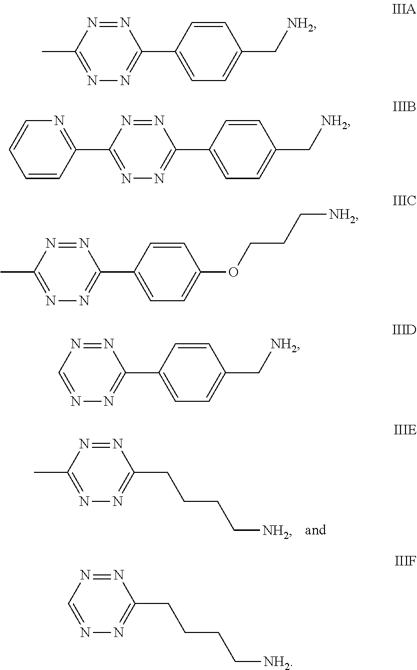

In some embodiments, each of the one or more linkers comprises the norbornene moiety covalently bound to the tetrazine moiety.

In some embodiments, any one of the norbornene moiety and the tetrazine moiety is covalently bound to (i) the first HA chain or (ii) to the second HA chain.

In some embodiments, the covalently bound is via a bond selected from the group comprising: amide, amine, ester, ether, carbamide, thiocarbamide, and carbamate.

In some embodiments, a molar ratio between (i) any one of: the norbornene moiety, the tetrazine moiety, or the styrene moiety; and (ii) the first HA chain, or the second HA chain is determined by ¹H NMR.

In one embodiment, the first HA chain and the second HA chain have an average molecular weight (MW) of 500,000 to 4,000,000 Daltons (Da).

In one embodiment, the polymer has a phase angle (δ) of 0.1 to 10°.

In one embodiment, the polymer has an elastic modulus (G′) of 10 to 1,000 Pa.

In one embodiment, a concentration of said polymer within said composition is between 4 and 20 mg/ml, or between 2 and 20 mg/ml.

In one embodiment, the sHA is characterized by an average molecular weight (MW) of less than 250 kDa.

In one embodiment, the sHA is characterized by water solubility of at least 0.1 g/L.

In one embodiment, the polymer is characterized by water solubility of at most 0.1 g/L.

In one embodiment, wherein at least 80% by weight of the first HA chain and of the second HA chain within the composition are cross-linked.

In one embodiment, the composition is characterized by an extrusion force of less than 30 N.

In some embodiments, the composition further comprises an acceptable carrier.

In some embodiments, the composition further comprises a compound selected from the group comprising: an amino acid, a mineral, a vitamin, an antioxidant, a nucleic acid, a coenzyme, an enzyme, a growth factor, a protein, an anti-tumor drug, a steroid, a non-steroidal anti-inflammatory drug, an antibiotic, an anesthetic agent, an antimicrobial drug, or any combination thereof.

In some embodiments, the composition is a pharmaceutical composition or a cosmeceutical composition.

In some embodiments, the composition is formulated for subcutaneous administration, topical administration or both.

In another aspect of the invention, there is provided a composition comprising (a) a polymer comprising a first hyaluronic acid (HA) chain or a derivative thereof and a second HA chain or a derivative thereof, wherein i. the first HA chain and the second HA chain are cross-linked via one or more linkers; ii. the one or more linkers comprise an unsaturated moiety or a derivative thereof and a tetrazine moiety or a derivative thereof; iii. the cross-linked is characterized by a crosslinking degree between 0.6% and 4%; and (b) an unmodified HA chain characterized by an average molecular weight (MW) between 300 kiloDaltons (kDa) and 4,000 kDa, wherein the composition is characterized by a phase angle (δ) up to 10°.

In some embodiments, the composition is characterized by being substantially devoid of particulate matter.

In some embodiments, the composition comprises between 5% and 30% weight per weight (w/w) of the unmodified HA chain.

In some embodiments, the composition is characterized by an average extrusion force between 5 newton (N) and 30 N.

In some embodiments, the composition is characterized by an extrusion profile devoid of peaks higher than 2 N.

In some embodiments, the composition is characterized by an elastic modulus (G′) between 20 Pa and 400 Pa.

In some embodiments, a concentration of the polymer within the composition is between 1 mg/ml and 25 mg/ml.

In some embodiments, the first HA chain and the second HA chain is characterized by an average MW between 500 kDa and 4,000 kDa.

In one embodiment, the unmodified HA is characterized by water solubility of at least 1 g/L.

In another aspect of the invention, there is provided a method for filling or volumizing a tissue in a subject in need thereof, comprising administering the composition of the present invention to the tissue, thereby filling or volumizing a tissue of a subject in need thereof.

In some embodiments, the tissue is selected from the group consisting of: skin, gingival, cartilage and ophthalmic tissue, muscles, and subcutaneous tissues.

In some embodiments, administering comprises subcutaneous or topical administration.

In another aspect of the invention, there is provided a process for manufacturing the composition of the present invention, the process comprising: mixing a first composition comprising the first HA chain of the present invention and a second composition comprising the second HA chain of the present invention, wherein the first composition, the second composition, or both comprises the unmodified HA chain of the present invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 include extrusion force curves of a composition according to Table 2, entry 1 (curve A), a composition according to Table 2, entry 2 (curve B) and a composition according to Table 2, entry 3 (curve C); and

FIGS. 2A-2D include pictures of dissolution experiment results of the gel in water after 30 minutes: according to procedure A (Table 1, Entry 4) (FIG. 2A), according to procedure B (Table 1, Entries 5-6) (FIGS. 2B-2C) and according to procedure C (Table 1, Entry 7) (FIG. 2D).

### DETAILED DESCRIPTION OF THE INVENTION

In some embodiments, the present invention is directed to a polymer comprising a first hyaluronic acid (HA) chain and a second HA chain crosslinked via a linker comprising an unsaturated moiety or a derivative thereof bound to a tetrazine moiety or a derivative thereof. In some embodiments, the polymer of the invention is characterized by a crosslinking degree of between 0.6 to 4%.

In some embodiments, the polymer of the invention (i.e. cross-linked HA) is substantially water insoluble. In some embodiments, the polymer of the invention is characterized by water solubility of at most 0.5 g/L, at most 0.3 g/L, at most 0.2 g/L, at most 0.1 g/L, at most 0.05 g/L, at most 0.01 g/L, at most 0.005 g/L, at most 0.001 g/L, including any range therebetween. In some embodiments, the terms “polymer” and “cross-linked HA” are used herein interchangeably.

In some embodiments, the present invention is directed to a polymer comprising a first hyaluronic acid (HA) chain and a second HA chain crosslinked via a linker comprising an unsaturated moiety or a derivative thereof bound to a tetrazine moiety or a derivative thereof. In some embodiments, the polymer of the invention is characterized by a crosslinking degree of between 0.6 to 4%.

In some embodiments, the polymer of the invention is substantially water insoluble.

Furthermore, the present invention in some embodiments thereof is based, in part, on the finding that compositions according to the present invention exhibit a desired stability as well as an improved homogeneity and improved mechanical properties, compared to a similar composition comprising the same components but obtained by different processes.

### Compositions

In some embodiments, there is a composition comprising the polymer of the invention (e.g. the water-insoluble polymer of the invention), wherein a weight ratio between water soluble HA (sHA) and the polymer within the composition is less than 10%, less than 8%, less than 6%, less than 5%, less than 3%, less than 2%, less than 1%, including any range therebetween. In some embodiments, sHA is as described hereinbelow. In some embodiments, the composition optionally comprises the polymer of the invention and/or a salt thereof. In some embodiments, the composition is substantially devoid of unmodified HA.

In exemplary embodiments, the unsaturated moiety is norbornene.

In some embodiments, the invention is directed to a polymer comprising a plurality of cross-linked hyaluronic acid (HA) chains or derivatives thereof. As used herein, HA chain or a derivative thereof comprises D-glucuronic acid and N-acetyl-glucosamine. In some embodiments, the polymer of the invention comprises two or more cross-linked HA chains. In some embodiments, the polymer of the invention comprises two or more cross-linked HA chains, wherein the polymer is characterized by a crosslinking degree of between 0.6 and 4%, between 0.6 and 0.8%, between 0.8 and 1%, between 0.6 and 1%, between 0.6 and 2%, between 1 and 1.5%, between 1.5 and 2%, between 2 and 4%, between 2 and 3%, including any range between. Each possibility represents a separate embodiment of the present invention.

In some embodiments, the crosslinking degree is as described hereinbelow.

As used herein, a derivative of HA chain relates to a chemically modified HA. In some embodiments, a chemically modified HA comprises a side chain modification (e.g. acetylation of a hydroxy group, decarboxylation, esterification or amidation of a carboxy group). In some embodiments, a chemically modified HA comprises one or more of the side chain modifications. In some embodiments, the modifications are the same. In some embodiments, the modifications are different. In some embodiments, a chemically modified HA comprises a combination of modified side chains.

In some embodiments, the concentration of the polymer within the composition of the invention is between 1 mg/ml and 25 mg/ml, between 4 and 20 mg/ml, between 4 and 6 mg/ml, between 6 and 8 mg/ml, between 2 and 8 mg/ml, between 2 and 10 mg/ml, between 2 and 20 mg/ml, between 6 and 8 mg/ml, between 8 and 10 mg/ml, between 10 and 15 mg/ml, between 15 and 20 mg/ml, including any range between. Each possibility represents a separate embodiment of the present invention.

In some embodiments, the first HA chain of the present invention comprises HA characterized by an average molecular weight between 500,000 and 1,000,000 Da, 500,000 and 1,500,000 Da, 500,000 and 5,000,000 Da, 750,000 and 4,000,000 Da, 1,000,000 and 7,500,000 Da, 2,000,000 and 10,000,000 Da, 1,000,000 and 2,000,000 Da, 2,000,000 and 4,000,000 Da, 650,000 and 8,000,000 Da, 4,000,000 and 10,000,000 Da, or between 7,500,000 and 15,000,000 Da, including any range therebetween. Each possibility represents a separate embodiment of the present invention.

In some embodiments, the second HA chain of the present invention comprises HA characterized by an average molecular weight between 500,000 and 1,000,000 Da, 500,000 and 1,500,000 Da, 500,000 and 5,000,000 Da, 750,000 and 4,000,000 Da, 1,000,000 and 7,500,000 Da, 2,000,000 and 10,000,000 Da, 1,000,000 and 2,000,000 Da, 2,000,000 and 4,000,000 Da, 650,000 and 8,000,000 Da, 4,000,000 and 10,000,000 Da, or between 7,500,000 and 15,000,000 Da, including any range therebetween. Each possibility represents a separate embodiment of the present invention.

As used herein, the term “molecular weight” encompasses any one of the average weight values selected from: Mn (Number average molar mass), NAMW (Number Average Molecular Weight), Mw (Mass average molar mass), WAMW (Weight Average Molecular Weight), Mz (Z average molar mass), Mv (Viscosity average molar mass), and MWCO (molecular weight cut-off). Unless stated otherwise this term refers to Mw.

In one embodiment, the invention is directed to a polymer comprising a polycarboxylated polysaccharide (e.g. hyaluronic acid), and/or a salt thereof, or a derivative thereof having a molecular weight in the ranges specified above.

In another aspect, the invention is directed to a composition comprising (a) a polymer comprising a first hyaluronic acid (HA) chain or a derivative thereof and a second HA chain or a derivative thereof, wherein: the first HA chain and the second HA chain are cross-linked via one or more linkers; the one or more linkers comprise an unsaturated moiety or a derivative thereof and a tetrazine moiety or a derivative thereof; the cross-linked is characterized by a crosslinking degree between 0.6% and 4%; and (b) an unmodified HA chain characterized by an average molecular weight (MW) between 300 kiloDaltons (kDa) and 4,000 kDa.

In some embodiments, the composition of the invention comprises the polymer of the invention and further comprising an unmodified HA chain characterized by an average molecular weight (MW) between 300 kiloDaltons (kDa) and 4,000 kDa.

In some embodiments, the composition is characterized by a phase angle (δ) up to 10°.

In some embodiments, the composition comprises the unmodified HA or unmodified HA chain, including any salt thereof. In some embodiments, the composition comprises a pharmaceutically acceptable salt of HA.

In some embodiments, the composition comprises the first HA chain and the second HA chain, including any salt thereof.

In some embodiments, the unmodified HA chain characterized by an average MW between 300 kDa and 4,000 kDa, 350 kDa and 4,000 kDa, 400 kDa and 4,000 kDa, 500 kDa and 4,000 kDa, 550 kDa and 4,000 kDa, 600 kDa and 4,000 kDa, 700 kDa and 4,000 kDa, 800 kDa and 4,000 kDa, 300 kDa and 2,500 kDa, 350 kDa and 2,500 kDa, 400 kDa and 2,500 kDa, 500 kDa and 2,500 kDa, 550 kDa and 2,500 kDa, 600 kDa and 2,500 kDa, 700 kDa and 2,500 kDa, 800 kDa and 2,500 kDa, 300 kDa and 1,000 kDa, 350 kDa and 1,000 kDa, 400 kDa and 1,000 kDa, 500 kDa and 1,000 kDa, 550 kDa and 1,000 kDa, 600 kDa and 1,000 kDa, 700 kDa and 1,000 kDa, or between 800 kDa and 1,000 kDa, including any range therebetween. Each possibility represents a separate embodiment of the present invention.

As used herein, an “unmodified HA chain” or a derivative thereof refers to any HA chain (i.e. pristine) devoid of any chemical modifications and not coupled to the polymer comprising a first hyaluronic acid (HA) chain or a derivative thereof and a second HA chain or a derivative thereof. In some embodiments, the unmodified HA chain refers to any HA chain devoid of any chemical modifications and not coupled to a tetrazine moiety, a norbornene moiety, and/or derivatives thereof.

In some embodiments, the composition comprises between 5% and 30% weight per weight (w/w), 7% and 30% (w/w), 8% and 30% (w/w), 10% and 30% (w/w), 5% and 25% (w/w), 7% and 25% (w/w), 8% and 25% (w/w), 10% and 25% (w/w), 5% and 20% (w/w), 7% and 20% (w/w), 8% and 20% (w/w), 10% and 20% (w/w), 5% and 15% (w/w), 7% and 15% (w/w), or between 8% and 15% (w/w) of the unmodified HA chain, including any range therebetween. Each possibility represents a separate embodiment of the present invention.

In some embodiments, the composition is characterized by being substantially devoid of particulate matter. As used herein, the term “particulate matter” refers to any particle (liquid droplets or solid) suspended in the mixture. In some embodiments, particulate matter refers to particles made of or comprising fractions of gel. In some embodiments, particulate matter refers to particles characterized by a size of less than 5 mm, less than 3 mm, less than 2 mm, or less than 1 mm, including any range therebetween. Each possibility represents a separate embodiment of the present invention.

In some embodiments, the composition is characterized by being substantially devoid of particles characterized by a size of less than 5 mm, less than 4 mm, less than 3 mm, less than 2 mm, less than 1 mm, or less than 0.5 mm, including any range therebetween. Each possibility represents a separate embodiment of the present invention. In some embodiments, the composition is characterized by being substantially devoid of particles characterized by a diameter of less than 5 mm, less than 4 mm, less than 3 mm, less than 2 mm, less than 1 mm, or less than 0.5 mm, including any range therebetween. Each possibility represents a separate embodiment of the present invention. In some embodiments, the particle diameter is an average particle diameter. In some embodiments, the particle size is an average particle size.

In some embodiments, the composition of the invention is characterized by a δ between 0.1° and 10°, 0.1° and 0.5°, 0.1° and 0.9°, 0.1° and 1°, 0.1° and 1.5°, 0.1º and 4.5°, 0.1° and 7.5°, 0.1° and 8.5°, 0.1 and 9.5°, 0.5° and 10°, 0.5° and 0.9°, 0.5° and 1°, 0.5° and 1.5°, 0.5° and 4.5°, 0.5° and 7.5°, 0.5° and 8.5°, 0.5 and 9.5°, 0.9° and 10°, 0.9° and 0.5°, 0.9° and 1°, 0.9° and 1.5°, 0.9° and 4.5°, 0.9° and 7.5°, or between 0.9° and 8.5°, including any range therebetween. Each possibility represents a separate embodiment of the present invention.

In some embodiments, the composition is characterized by a δ of 0.1° at most, 0.5° at most, 0.7° at most, 0.9° at most, 1° at most, 1.5° at most, 2° at most, 2.7° at most, 3.2° at most, 4° at most, 4.5° at most, 6° at most, 7.5° at most, 8° at most, 9° at most, or 10° at most, including any value therebetween. Each possibility represents a separate embodiment of the present invention.

In some embodiments, the composition is characterized by an extrusion profile devoid of peaks higher than 2 N, higher than 1.9 N, higher than 1.8 N, higher than 1.7 N, higher than 1.5 N, or higher than 1 N, including any value therebetween. Each possibility represents a separate embodiment of the present invention.

In some embodiments, the composition is characterized by a phase angle (δ) up to 10°, up to 9.5°, up to 9°, up to 8.5°, up to 8°, up to 7.5°, or up to 7°, including any value therebetween. Each possibility represents a separate embodiment of the present invention.

As used herein, the term “phase angle” or “δ” refers to degree of viscoelasticity of a material. As would be apparent to one of ordinary skill in the art, δ can be calculated according to the following equation:

δ=Inverse Tangent

$$( \frac{G ″}{G ′} ) ;$$

wherein G″ is the viscosity modulus and G′ is the elasticity modulus. In some embodiments, G′ and G″ are obtained by oscillatory rheology and are measured in the viscoelastic domain for low oscillation in stress or amplitude.

In some embodiments, the composition of the invention is elastic. As used herein, the elasticity of a polymer is characterized by the elastic modulus (G′). In some embodiments, the term “elastic modulus” means the elastic modulus as determined hereinbelow.

In some embodiments, the composition is characterized by an elastic modulus (G′) between 20 Pa and 400 Pa, 40 Pa and 400 Pa, 45 Pa and 400 Pa, 50 Pa and 400 Pa, 70 Pa and 400 Pa, 80 Pa and 400 Pa, 100 Pa and 400 Pa, 45 Pa and 350 Pa, 50 Pa and 350 Pa, 70 Pa and 350 Pa, 80 Pa and 350 Pa, 95 Pa and 350 Pa, 150 Pa and 350 Pa, 48 Pa and 250 Pa, 55 Pa and 250 Pa, 70 Pa and 250 Pa, 80 Pa and 250 Pa, 90 Pa and 250 Pa, or between 100 Pa and 250 Pa, including any range therebetween. Each possibility represents a separate embodiment of the present invention.

In some embodiments, the composition of the invention is an injectable composition. In some embodiments, the composition of the invention is an extrudable composition. In some embodiments, the composition of the invention is characterized by an extrusion force of less than 30 newton (N), less than 25 N, less than 20 N, less than 15 N, or less than 10 N including any range between. Each possibility represents a separate embodiment of the present invention.

As used herein, the term “extrusion force” is related to a force required for extrusion of the composition of the invention from a syringe via 27G ½″ needle, or a needle having a diameter of less than 27G ½″. The extrusion force can be measured according to a method described herein (see Examples section).

In some embodiments, the composition of the invention is characterized by an average extrusion force between 5 N and 30 N, 7 N and 30 N, 8 N and 30 N, 10 N and 30 N, 5 N and 20 N, 7 N and 20 N, 8 N and 20 N, 10 N and 20 N, 5 N and 18 N, 7 N and 18 N, 8 N and 18 N, 10 N and 18 N, 5 N and 15 N, 7 N and 15 N, 8 N and 15 N, or between 10 N and 15 N, including any range therebetween. Each possibility represents a separate embodiment of the present invention.

In some embodiments, the composition of the invention is characterized by a maximum extrusion force between 5 N and 30 N, 7 N and 30 N, 8 N and 30 N, 10 N and 30 N, 5 N and 20 N, 7 N and 20 N, 8 N and 20 N, 10 N and 20 N, 5 N and 18 N, 7 N and 18 N, 8 N and 18 N, 10 N and 18 N, 5 N and 15 N, 7 N and 15 N, 8 N and 15 N, or between 10 N and 15 N, including any range therebetween. Each possibility represents a separate embodiment of the present invention.

In some embodiments, the concentration of the polymer within the composition of the invention is between 1 mg/ml and 25 mg/ml, between 4 and 20 mg/ml, between 4 and 6 mg/ml, between 6 and 8 mg/ml, between 8 and 10 mg/ml, between 10 and 15 mg/ml, between 15 and 20 mg/ml, including any range between. Each possibility represents a separate embodiment of the present invention.

In some embodiments, the unmodified HA is substantially water soluble. In some embodiments, the unmodified HA is characterized by water solubility of at least 1 g/L, at least 2 g/L, at least 5 g/L, at least 10 g/L, at least 20 g/L, at least 50 g/L, including any range between. Each possibility represents a separate embodiment of the present invention.

In some embodiments, the unmodified HA is characterized by water solubility of between 0.5 g/L and 10 g/L, between 0.5 g/L and 1 g/L, between 1 g/L and 10 g/L, between 10 g/L and 30 g/L, between 30 g/L and 50 g/L, between 50 g/L and 100 g/L, including any range between.

In some embodiments, the composition is substantially devoid of low MW soluble HA (sHA).

As used herein, the term “low MW sHA” refers to water soluble polymers and/or oligomers of HA characterized by a MW of less than 300 kDa, less than 250 kDa, less than 200 kDa, less than 150 kDa, less than 100 kDa, less than 80 kDa, less than 50 kDa, less than 20 kDa, less than 10 kDa, less than 1 kDa, including any range between. Each possibility represents a separate embodiment of the present invention. In some embodiments, the low MW sHA is substantially devoid of chemically modified HA (e.g. the first HA chain and/or the second HA chain of the invention). The terms “low MW sHA” and “sHA” are used herein interchangeably.

In some embodiments, the composition is substantially devoid of sHA characterized by a MW of less than 250 kDa, less than 200 kDa, less than 150 kDa, less than 100 kDa, less than 80 kDa, less than 50 kDa, less than 20 kDa, less than 10 kDa, less than 1 kDa, including any range between. Each possibility represents a separate embodiment of the present invention.

In some embodiments, the composition comprises between 0.01% and 10%, 0.05% and 10%, 0.1% and 10%, 0.5% and 10%, 0.9% and 10%, 1% and 10%, 0.01% and 9.8%, 0.05% and 9.8%, 0.1% and 9.8%, 0.5% and 9.8%, 0.9% and 9.8%, or between 1% and 9.8% of low MW sHA, including any range therebetween. Each possibility represents a separate embodiment of the present invention.

In some embodiments, the composition comprises between 0.01% and 30%, 0.05% and 10%, 0.1% and 10%, 0.5% and 10%, 0.9% and 10%, 1% and 10%, 0.01% and 9.8%, 0.05% and 9.8%, 0.1% and 9.8%, 0.5% and 9.8%, 0.9% and 9.8%, 10 and 20%, 20 and 30%, or between 1% and 9.8% of low MW sHA, including any range therebetween. Each possibility represents a separate embodiment of the present invention.

In some embodiments, the low MW sHA content comprises a weight portion of the sHA from the soluble (e.g. water soluble and/or water extractable) HA content. In some embodiments, low MW sHA is as described hereinbelow. The content of the low MW sHA within the soluble HA fraction can be determined according to a procedure (e.g. HPLC) described herein (see Examples section).

In some embodiments, the sHA content comprises a weight portion of the sHA from the soluble HA content. In some embodiments, sHA is as described hereinbelow. The content of the sHA within the soluble HA fraction can be determined according to a procedure (e.g. HPLC) described herein (see Examples section).

In some embodiments, the low MW sHA comprises water soluble HA chains. In some embodiments, water solubility of the low MW sHA is at least 1 g/L, at least 2 g/L, at least 5 g/L, at least 10 g/L, at least 20 g/L, at least 50 g/L, including any range between. Each possibility represents a separate embodiment of the present invention.

In some embodiments, the low MW sHA is characterized by water solubility of between 0.5 g/L and 10 g/L, between 0.5 g/L and 1 g/L, between 1 g/L and 10 g/L, between 10 g/L and 30 g/L, between 5 g/L and 30 g/L, between 5 g/L and 20 g/L, between 30 g/L and 50 g/L, between 50 g/L and 100 g/L, including any range between. In some embodiments, the low MW sHA is characterized by water solubility of between 5 g/L and 20 g/L, including any range between. Each possibility represents a separate embodiment of the present invention.

In some embodiments, the sHA comprises water soluble HA chains. In some embodiments, water solubility of the sHA is at least 1 g/L, at least 2 g/L, at least 5 g/L, at least 10 g/L, at least 20 g/L, at least 50 g/L, including any range between. Each possibility represents a separate embodiment of the present invention.

In some embodiments, the sHA is characterized by water solubility of between 0.5 g/L and 10 g/L, between 0.5 g/L and 1 g/L, between 1 g/L and 10 g/L, between 10 g/L and 30 g/L, between 5 g/L and 30 g/L, between 5 g/L and 20 g/L, between 30 g/L and 5 0 g/L, between 50 g/L and 100 g/L, including any range between. In some embodiments, the sHA is characterized by water solubility of between 5 g/L and 20 g/L, including any range between. Each possibility represents a separate embodiment of the present invention.

### Polymer

In some embodiments, the polymer of the present invention comprises one or more HA chains. In some embodiments, “one or more” is two. In some embodiments, two HA chains of the invention are cross-linked. In one embodiment, cross-linking is inter-crosslinking. As defined herein, the term “inter” refers to the formation of a bond between two moieties residing in two different chains, as opposed to the formation of an “intra” bond between two residues residing within the same chain. In one embodiment, cross-linking is intra-crosslinking. In some embodiments, crosslinking of two HA chains is via a linker.

A “linker” as defined herein refers to a molecule or macromolecule serving to connect different moieties or functional groups of one or more polycarboxylated polysaccharides. In one embodiment, a linker may also facilitate other functions, including, but not limited to, preserving biological activity, maintaining interactions, and others.

In some embodiments, the polymer of the present invention comprises a first HA chain cross-linked with, or covalently linked to a second HA chain via one or more linkers, wherein the one or more linkers comprises a compound represented by Formula (A) and/or by Formula (B):

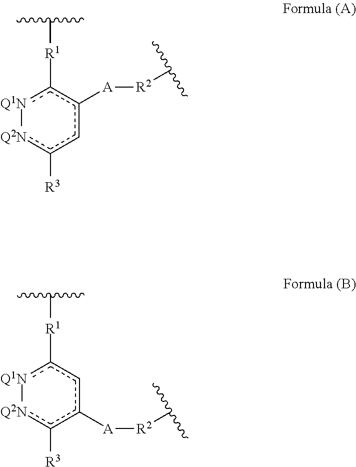

or a combination thereof; wherein: [symbol] represents a single or a double bond; R¹, R², or both, are selected from the group consisting of: a bond, alkyl, alkenyl, cycloalkyl, aryl, heteroaryl, heteroalicyclic, C(O)—NH-alkyl-NH, and alkyl-NZ, or a combination thereof, wherein Z is a bond, aryl, or heteroaryl; A is selected form the group consisting of: a bond, alkyl, and aryl; Q¹, Q² or both represent hydrogen, or are absent; and wherein R³ is selected from the group consisting of: hydrogen, alkyl, aryl, or heteroaryl substituted or non-substituted. In some embodiments, a wavy bond represents HA.

In some embodiments, the polymer of the present invention comprises a first HA chain connected to a second HA chain by one or more linkers comprising a compound represented by

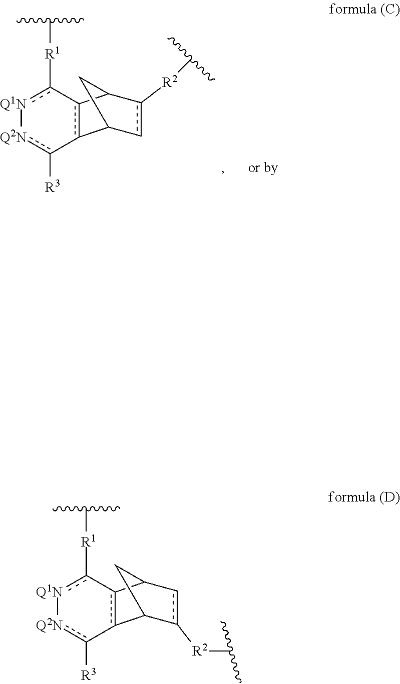

or a combination thereof; wherein: [symbol] represents a single or a double bond and wherein Q¹, Q², R¹, R², and R³ are as described hereinabove. In some embodiments, a wavy bond represents HA.

In some embodiments, the polymer of the present invention comprises a first HA chain cross-linked to a second HA chain via a linker, wherein the linker is represented by formula (C) or (D).

In some embodiments, the linker comprises a first compound derived from an unsaturated moiety and a second compound derived from a tetrazine moiety (e.g. comprising a tetrazine derivative, such as pyridazine). In some embodiments, the first compound and the second compound are covalently bound. In some embodiments, the first compound and the second compound are covalently bound, thereby forming a compound represented by any one of formulae (A) to (D).

In some embodiments, R¹, R², and R³, each independently comprises a substituent selected from the group consisting of: alkyl, cycloalkyl, aryl, heteroalicyclic, heteroaryl, alkoxy, hydroxy, phosphonate, thiohydroxy, thioalkoxy, aryloxy, thioaryloxy, amino, nitro, halo, trihalomethyl, cyano, amide, amine, alkanoamine, carboxy, sulfonyl, sulfoxy, sulfinyl, and sulfonamide.

In some embodiments, the polymer of the present invention comprises a first HA chain connected to a second HA chain via a linker, wherein the linker is or comprises any one of:

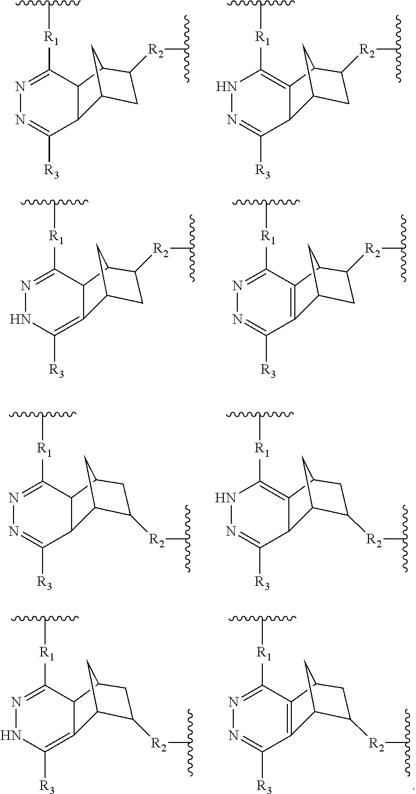

In some embodiments, R¹ is selected from the group comprising: -C₀-C₆alkyl-NZ—, -C₀-C₆alkyl-O—, and -C₀-C₆alkyl-C(O)—.

In some embodiments, Z is selected from the group comprising: a bond, aryl, or heteroaryl, wherein the aryl and heteroaryl are optionally substituted with halogen, hydroxy, C₁-C₆alkyl, C₁-C₆alkoxy, (C₁-C₆alkyl) amino, and di(C₁-C₆alkyl)amino;

In some embodiments, R² is selected from the group comprising: C₀-C₆alkyl-NZ, -C₀-C₆alkyl O—, and C₀-C₆alkyl-C(O)—.

In some embodiments, R³ is selected from the group comprising: hydrogen, C₁-C₆alkyl, aryl, or heteroaryl, wherein the aryl and heteroaryl are optionally substituted with halogen, hydroxy, C₁-C₆ alkyl, C₁-C₆ alkoxy, (C₁-C₆-alkyl)amino, and di(C₁-C₆alkyl)amino.

In some embodiments, R¹,R², or both are selected from the group comprising: —NZ—, -C₁-C₆ alkyl-NZ—, —O—, -C₁-C₆ alkyl-O—, —C(O)—, or -C₁-C₃alkyl-C (O)—; -methyl-O ,-pentyl-O—; —C(O)—; and -methyl-C(O)—.

In some embodiments, Z is a bond. In some embodiments, Z is selected from the group comprising: aryl and heteroaryl, phenyl; pyridyl, pyrimidyl, and pyrazinyl; each optionally may be substituted.

In some embodiments, the linker is or comprises a compound represented by any of formulae (C) or (D) according to any preceding embodiment, wherein R¹, R² or both are selected from the group comprising: —NZ—, -C₁-C₆-alkyl-NZ—, —O—, -C₁-C₆ alkyl —O—, —C(O)—, or -C₁-C₃-alkyl-C(O)—; -C₁-C₆ alkyl-NZ—; C₁-C_{3,} alkyl-NZ—; —methyl-NH— or -pentyl-NH—; -C₁-C₆ alkyl-O—; -C₁-C₃ alkyl-O—; -methyl-O, or -pentyl-O—; -C₀-C₃ alkyl-C(O)—; C(O)—; and -methyl-C(O)—.

In some embodiments, R³ is hydrogen.

In some embodiments, R³ is selected from the group comprising: C₁-C₆ alkyl, aryl, or heteroaryl, wherein the aryl or the heteroaryl may be optionally substituted; aryl or heteroaryl, wherein aryl and heteroaryl are optionally substituted; phenyl; pyridyl, pyrimidyl, or pyrazinyl.

In some embodiments, the linker of the invention is represented by formula (C) or by formula (D), wherein R³ is selected from the group comprising: C₁-C₆ alkyl, C₁-C₃ alkyl, and methyl or a combination thereof.

In some embodiments, the polymer comprises the first HA chain and the second HA chain cross-linked via a plurality of linkers comprising a compound represented by formula (C) or by formula (D).

In some embodiments, the linker of the invention is formed via a reaction between the first HA chain and the second HA chain. In some embodiments, the reaction is a cyclization reaction and/or a cycloaddition reaction. In some embodiments, the linker of the invention is formed via a reaction between the unsaturated moiety and the tetrazine moiety. In some embodiments, the linker of the invention is formed via a cyclization reaction and/or a cycloaddition reaction between the unsaturated moiety and the tetrazine moiety. In some embodiments, the linker of the invention is derived from the unsaturated moiety and from the tetrazine moiety. In some embodiments, the linker of the invention is derived from the norbornene moiety and from the tetrazine moiety.

In some embodiments, the polymer of the invention comprises a first HA chain covalently bound to a second HA chain via a linker, wherein the linker is obtained via a cyclization reaction. In some embodiments, the linker is obtained via a cycloaddition reaction. Various cyclization and/or cycloaddition reactions are well-known in the art (such as a photochemical cycloaddition, Diels-Alder cyclization, or any other chemical reaction resulting in the formation of a cyclic ring). In some embodiments, the cyclization and/or cycloaddition reaction is or comprises an inverse electron-demand Diels-Alder cyclization of an unsaturated moiety (e.g. norbornene moiety) or a derivative thereof, and a tetrazine moiety or a derivative thereof.

In some embodiments, a first HA chain of the invention comprises an unsaturated moiety, as described herein above. In some embodiments, a first HA chain comprises the unsaturated moiety bound to the HA. In some embodiments, a first HA chain comprises the unsaturated moiety covalently bound to the HA. In some embodiments, a first HA chain of the invention comprises a styrene moiety or a derivative thereof.

In some embodiments, a styrene moiety is represented by formulae IA-D:

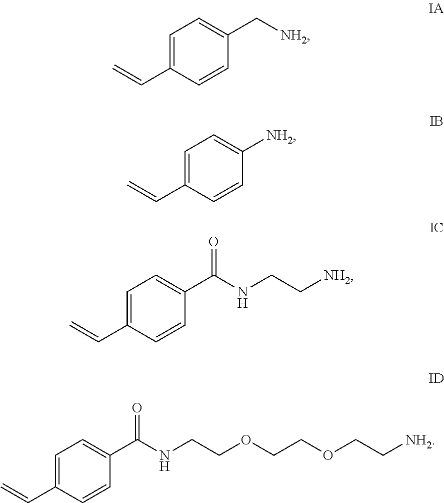

In some embodiments, a derivative of the styrene moiety comprises the styrene moiety of formulae IA-ID bound to the first HA chain via a covalent bond.

Non-limiting examples of covalent bonds include, but are not limited to: amide, amine, ester, ether, carbamide, thiocarbamide, and carbamate.

In some embodiments, the amine of the styrene moiety is covalently bound to the first HA chain. In some embodiments, the styrene moiety is bound to a carboxy group of the first HA chain. In some embodiments, a derivative of the styrene moiety is referred to a styrene moiety (e.g. compound of formula IA) bound to the first HA chain via an amide bond, as represented by formula IE:

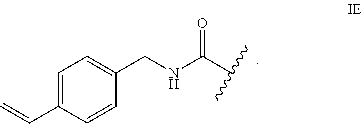 [SMILES: *C(=O)NCc1ccc(C=C)cc1]

In some embodiments, a first HA chain of the invention comprises HA covalently bound to the styrene moiety (e.g. of Formula IA) via an amide bond.

In some embodiments, the unsaturated moiety is or comprises a norbornene moiety, or a derivative thereof. In some embodiments, a first HA chain of the invention comprises a norbornene moiety, or a derivative thereof covalently bound to the HA. In some embodiments, the number of norbornene moieties or derivatives thereof covalently bound to the first HA chain of the invention may be any integer between 1 and 100,000, including any range between.

In some embodiments, a second HA chain of the invention comprises a tetrazine moiety, or a derivative thereof covalently bound to the HA. In some embodiments, the number of tetrazine moieties or derivatives thereof covalently attached to the second HA chain of the invention ranges between 1 and 100,000 including any range between.

In some embodiments, at least a portion of the first HA chain and/or of the second HA chain is covalently bound to a tetrazine moiety or to a norbornene moiety, or a derivative thereof.

In some embodiments, the composition of the invention comprises between 10 and 99%, between 10 and 20%, between 20 and 50%, between 50 and 70%, between 70 and 80%, between 80 and 90%, between 90 and 99%, including any range between of the first HA chains and/or of the second HA chains covalently bound to any of (i) tetrazine moiety; (ii) norbornene moiety and/or styrene moiety, or a derivative thereof.

In some embodiments, each of the first HA chains comprises, 1-10,000, 1-5,000, 1-1,000, 5,000-50,000, 5,000-10,000, 1,000-10,000, 1,000-5,000, 500-5,000, 500-1000, or 1-500 norbornene moieties or derivatives thereof, including any range between. In some embodiments, each of the second HA chains comprises 1-100,000, 1-50,000, 1-10,000, 1-5,000, 1-1,000, 5,000-50,000, 5,000-10,000, 1,000-10,000, 1,000-5,000, 500-5,000, 500-1,000, or 1-500 tetrazine moieties or derivatives thereof, including any range between. Each possibility represents a separate embodiment of the present invention.

In some embodiments, a norbornene moiety is in the endo conformation, and/or exo conformation. Non-limiting examples of norbornene moieties include, but are not limited to compounds represented by any of formulae IIA-IIG:

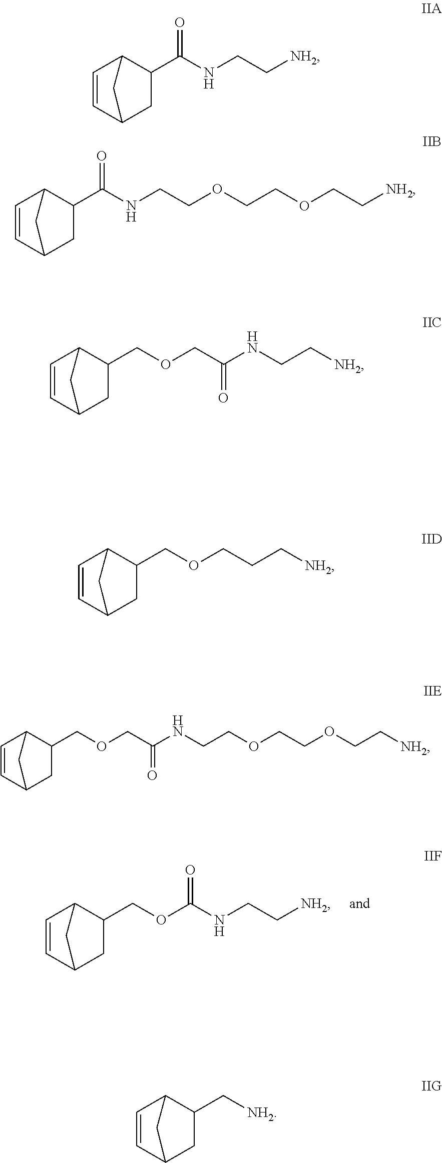

Non-limiting examples of tetrazine moieties include, but are not limited to compounds represented by any of formulae IIIA-IIIF:

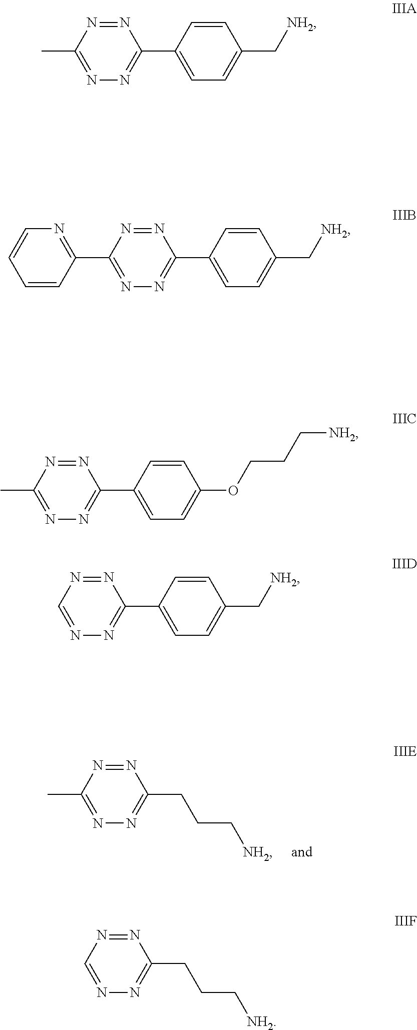 [SMILES: Cc1nnc(-c2ccc(CN)cc2)nn1 ; NCc1ccc(-c2nnc(-c3ccccn3)nn2)cc1 ; Cc1nnc(-c2ccc(OCCCN)cc2)nn1 ; NCc1ccc(-c2nncnn2)cc1 ; Cc1nnc(CCCN)nn1 ; NCCCc1nncnn1]

In some embodiments, the norbornene moiety is or comprises the compound of formula IIA:

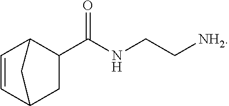 [SMILES: NCCNC(=O)C1CC2C=CC1C2]

In some embodiments, a derivative of the norbornene moiety comprises a norbornene moiety represented by any of formulae IIA-IIG, bound to the first HA chain via a covalent bond. In some embodiments, the covalent bond is selected from amide, amine, ester, ether, carbamide, thiocarbamide, and carbamate.

In some embodiments, the amine of the norbornene moiety is bound to HA of the first HA chain. In some embodiments, the norbornene moiety is bound to the carboxy group of the HA. In some embodiments, a derivative of the norbornene moiety is referred to a norbornene moiety (e.g. a compound of formula IIA) bound to the HA via an amide bond, as represented by formula IF:

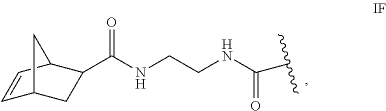 [SMILES: *C(=O)NCCNC(=O)C1CC2C=CC1C2]

wherein a wavy bond represents HA.

In some embodiments, the norbornene moiety is bound to the HA of the first HA chain via an amide bond or an ester bond. In some embodiments, the amide bond or an ester bond is formed by reaction of the HA and the norbornene moiety, optionally with an appropriate coupling agent.

In some embodiments, the norbornene moiety is bound to the HA of the first HA chain by reacting the HA and the norbornene moiety (e.g. any of Formulae IIA-IIG) with an appropriate coupling agent. In some embodiments, the coupling agent comprises any compound capable of catalyzing the reaction between the carboxy group of the HA and the nucleophilic group of the norbornene moiety (e.g. an amine or a hydroxy group).

Non-limiting examples of coupling agents include, but are not limited to, 1-ethyl-3-(3-dimethylamino-propyl)carbodiimide (EDC), carbonyl diimidazole, N,N′-dicyclohexylcarbodiimide (DCC), N,N′-diisopropylcarbodiimide (DIC), 4-(4,6-dimethoxy-1,3,5-triazin-2-yl)-4-methyl-morpholinium chloride (DMTMM) and N,N,N′,N′-tetramethyl-O-(1H benzotriazol-1-yl)uronium hexafluorophosphate (HBTU).

In one embodiment, a derivative of the tetrazine moiety is related to the tetrazine moiety, as defined herein above, is bound to the HA of the second HA chain via a covalent bond. In some embodiments, the covalent bond is as defined herein above.

In some embodiments, a derivative of the tetrazine moiety comprises a tetrazine moiety represented by any of formulae IIIA-IIIF bound to the HA of the second HA chain via a covalent bond. In some embodiments, the covalent bond is selected from amide, amine, ester, ether, carbamide, thiocarbamide, and carbamate.

In some embodiments, the amine of the tetrazine moiety is bound to the HA of the second HA chain. In some embodiments, the tetrazine moiety is bound to the carboxy group of the HA. In some embodiments, a derivative of the tetrazine moiety is referred to a tetrazine moiety (e.g., a compound of formula IIIB) bound to the HA via an amide bond, as represented by formula IG:

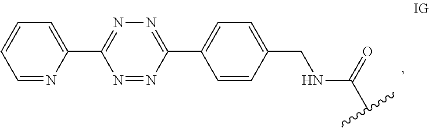 [SMILES: *C(=O)NCc1ccc(-c2nnc(-c3ccccn3)nn2)cc1]

wherein a wavy bond represents HA.

In some embodiments, the tetrazine moiety is bound to the HA of the second HA chain via an amide bond or an ester bond. In some embodiments, the amide bond or an ester bond is formed by reaction of the HA and the norbornene moiety, optionally with an appropriate coupling agent.

In some embodiments, the tetrazine moiety is bound to the HA of the second HA chain by reacting the HA and the tetrazine moiety (e.g. benzyl amine tetrazine), with an appropriate coupling agent, wherein the coupling agent is as described herein.

In some embodiments, the norbornene moiety or a derivative thereof coupled to the HA of the first HA chain; and the tetrazine moiety or a derivative thereof coupled to the HA of the second HA chain, are present within the polymer of the invention at a ratio of 3:1, 3:2, 2:1, 1:1, 1:2, 2:3, or 1:3, including any value and range there between. Each possibility represents a separate embodiment of the present invention. As defined herein, ratio is any ratio selected from: molar ratio, weight ratio, or concentration ratio.

As defined herein, the term “crosslinking degree” refers to the molar ratio between the linker and the repeating unit of the carboxylated polysaccharide forming the polymer of the invention. In some embodiments, the terms “crosslinking degree” and “modification degree” (of the first HA chain and/or of the second HA chain) are used herein interchangeably. It is assumed that the norbornene moiety or a derivative thereof of the first HA chain and the tetrazine moiety or a derivative thereof of the second HA chain react almost completely with each other (about 90-100% cross-linking yield), accordingly the crosslinking degree is almost completely predominated by the modification degree of the first HA chain and/or of the second HA chain. In some embodiments, the first HA chain and of the second HA chain have substantially the same modification degree.

In some embodiments, the crosslinking degree refers to the molar ratio between the pristine (or non-reacted) norbornene moiety or tetrazine moiety of the first HA chain or of the second HA chain, respectively; and the repeating unit of the carboxylated polysaccharide forming the polymer of the invention. In some embodiments, the crosslinking degree refers to the molar ratio between the linker and the repeating unit of the polymer of the invention. In some embodiments, the crosslinking degree refers to the molar ratio between the linker and the repeating unit of the HA. In some embodiments, the crosslinking degree refers to a ratio between the number of linkers within the polymer of the invention and the initial number of carboxy groups in the pristine HA (e.g., also referred to herein as the modification degree) prior to reaction thereof with the unsaturated moiety or with the tetrazine moiety of the invention. In some embodiments, the crosslinking degree refers to a ratio between the number of linkers within the polymer of the invention and the median number of repeating units (or monomers) of the HA.

In some embodiments, the modification degree of the first HA chain (e.g. a molar ratio between the unsaturated moieties such as norbornene moieties or styrene moieties and the carboxy groups of the pristine HA chain) or the modification degree of the second HA chain (e.g. a molar ratio between the tetrazine moieties and the carboxy groups of the pristine HA chain) each independently is between 0.6% and 3%, 0.7% and 3%, 0.8% and 3%, 0.6% and 4%, 0.6% and 3.5%, 0.6% and 3.2%,0.6% and 2.5%, 0.7% and 4%, 0.7% and 3.5%, 0.7% and 2.5%, 0.7% and 2.7%, 0.7% and 3.2%, 0.8% and 3.5%, 0.8% and 4%, 0.8% and 2.5%, 0.8% and 2.7%, 0.8% and 3.2%, 0.6% and 2.5%, 0.6% and 1%, 0.6% and 1.75%, 1% and 2%, 1% and 2.5%, 2% and 2.5%, 2.25% and 3%, 2.5% and 3.25%, 3% and 3.75%, or between 3.6% and 4%, including any range therebetween.

In some embodiments, the crosslinking degree of the polymer of the invention is 0.6% at most, 0.7% at most, 0.9% at most, 1% at most, 2% at most, 3% at most, or 4% at most, including any value therebetween. In some embodiments, the crosslinking degree of a polymer of the invention is between 0.6% and 3%, 0.7% and 3%, 0.8% and 3%, 0.6% and 4%, 0.6% and 3.5%, 0.6% and 3.2%,0.6% and 2.5%, 0.7% and 4%, 0.7% and 3.5%, 0.7% and 2.5%, 0.7% and 2.7%, 0.7% and 3.2%, 0.8% and 3.5%, 0.8% and 4%, 0.8% and 2.5%, 0.8% and 2.7%, 0.8% and 3.2%, 0.6% and 2.5%, 0.6% and 1%, 0.6% and 1.75%, 1% and 2%, 1% and 2.5%, 2% and 2.5%, 2.25% and 3%, 2.5% and 3.25%, 3% and 3.75%, or between 3.6% and 4%, including any range therebetween. Each possibility represents a separate embodiment of the present invention. In one embodiment, the crosslinking degree of a polymer refers to the calculated mean of crosslinking degree of a plurality of HA chains within a polymer of the invention.

In some embodiments, the composition of the invention comprises: (i) the polymer of the invention characterized by a crosslinking degree between 0.6% and 4%, between 0.6% and 3%, 0.7% and 3%, 0.8% and 3%, 0.6% and 4%, 0.6% and 3.5%, 0.6% and 3.2%,0.6% and 2.5%, 0.7% and 4%, 0.7% and 3.5%, 0.7% and 2.5%, 0.7% and 2.7%, 0.7% and 3.2%, 0.8% and 3.5%, 0.8% and 4%, 0.8% and 2.5%, 0.8% and 2.7%, 0.8% and 3.2%, including any range therebetween; and (ii) the unmodified HA chain characterized by an average molecular weight (MW) between 300 kiloDaltons (kDa) and 4,000 kDa, and wherein the composition is characterized by a phase angle (δ) up to 10°. In some embodiments, the composition of the invention consists essentially of the polymer of the invention including any salt thereof, and of the unmodified HA chain including any salt thereof.

In some embodiments, the composition of the invention comprises the polymer of the invention characterized by a crosslinking degree between 0.6% and 4%, between 0.6% and 3%, 0.7% and 3%, 0.8% and 3%, 0.6% and 4%, 0.6% and 3.5%, 0.6% and 3.2%,0.6% and 2.5%, 0.7% and 4%, 0.7% and 3.5%, 0.7% and 2.5%, 0.7% and 2.7%, 0.7% and 3.2%, 0.8% and 3.5%, 0.8% and 4%, 0.8% and 2.5%, 0.8% and 2.7%, 0.8% and 3.2%, including any range therebetween; wherein the composition optionally comprises sHA, and wherein a weight ratio between the sHA and the polymer of the invention within the composition is less than 10%. In some embodiments, the composition of the invention consists essentially of the polymer of the invention including any salt thereof.

In some embodiments, the polymer of the invention is substantially water insoluble. In some embodiments, the polymer of the invention is characterized by water solubility of at most 0.5 g/L, at most 0.3 g/L, at most 0.2 g/L, at most 0.1 g/L, at most 0.05 g/L, at most 0.01 g/L, at most 0.005 g/L, at most 0.001 g/L, including any range therebetween. Each possibility represents a separate embodiment of the present invention.

In some embodiments, the polymer of the invention and/or the composition comprising thereof (e.g. a composition devoid of unmodified HA) has a phase angle (δ) of 0.1-25°, 0.1-0.5°, 0.1-0.9°, 0.5-1°, 0.7-1.5°, 1-2.5°, 2-4.5°, 3-4.75°, 4.7-5.5°, 5-7.5°, 6-8°, 7-8.5°, 8.25-9.5°, 9-10.5°, 9.5-12°.

In some embodiments, the polymer of the present invention and/or the composition comprising thereof (e.g. a composition devoid of unmodified HA) has an elastic modulus of 10-500 Pa, 20-1,000 Pa, 30-600 Pa, 40-1,000 Pa, 40-5,000 Pa, 50-10,000 Pa, 500-50,000 Pa, 500-10,000 Pa, 500-5,000 Pa, 500-1,000 Pa, 1,000-50,000 Pa, 1,000-10,000 Pa, 1,000-5,000 Pa, 50-20,000 Pa, 500-20,000 Pa, or 1,000-20,000 Pa. In some embodiments, the polymer of the present invention has an elastic modulus up to 10,000 Pa, up to 20,000 Pa, up to 30,000 Pa, up to 40,000 Pa, or up to 50,000 Pa. Each possibility represents a separate embodiment of the present invention.

### Manufacturing Process

In some embodiments, the present invention is directed to a process of preparing the polymer of the invention, the process comprising the steps of: (i) linking an unsaturated moiety (e.g. norbornene moiety) (or a derivative thereof) to a first hyaluronic acid (HA) chain (or a derivative thereof) and linking a tetrazine moiety (or a derivative thereof) to a second HA chain (or a derivative thereof); (ii) mixing the first (HA) chain (or a derivative thereof) comprising the norbornene moiety (or a derivative thereof) and the second HA chain (or a derivative thereof) comprising the tetrazine moiety (or a derivative thereof); and (iii) crosslinking the first HA chain to the second HA chain.

In some embodiments, the step (i) further comprises purifying the HA chain linked to the unsaturated moiety or to the tetrazine moiety.

Following the attachment of the unsaturated moiety or the tetrazine moiety to the HA chain, the resulting product is purified from unreacted starting materials, as well as well as from other reaction side products. Purification can be performed by any one of the methods known in the art, such as dialysis, precipitation, ultrafiltration or tangential flow filtration.

In some examples, the crosslinking reaction occurs at a usable range of temperature and conditions for forming cross-linked polymers and occurs without the input of external energy. In one embodiment, the crosslinking reaction is heated to increase reaction efficacy.

In some embodiments, the process for preparing the polymer of the invention comprises a spontaneous cross-linking reaction. In some embodiments, by “spontaneous chemical reaction” it is meant to refer to a process that is not assisted by e.g., light, heat, or radicals. In some embodiments, the crosslinking reaction may occur in water, in aqueous buffers or in cell culture media. Non-limiting examples for culture media include, but are not limited to, phosphate buffered saline, Hank's balanced salt solution, Dulbecco's Modified Eagle Medium, and the like. In some embodiments, the cross-linking can occur in organic solvents. Non-limiting examples for organic solvents include, but are not limited to methanol, ethanol, dichloromethane, dimethylformamide, and the like.

In some embodiments, the cross-linking reaction can occur at a wide range of temperatures of at least −80° C., at least −50° C., at least −20° C., at least 0° C., at least 4° C., at least 22° C., at least 37° C., or at least 45° C., and not more than 65° C. In some embodiments, the cross-linking reaction can occur at a wide range of temperatures of (−80)-(−50)° C., (−60)-(−15)° C., (−20)-(−4)° C., (−5)-0° C., (−2)-4° C., 2-8° C., 5-20° C., 15-30° C., 25-40° C., or 35-55° C.

In some embodiments, the steps (ii) and (iii) are performed in-situ, so as to form the cross-linked polymer by mixing the first (HA) chain and the second (HA) chain. In some embodiments, the preparation process is devoid of a post-processing step such as sieving or homogenization.

In some embodiments of the preparation process, after the cross-linking step, unreacted norbornene or a derivative thereof and/or tetrazine or a derivative thereof may remain attached to the HA chains. As defined herein, the term “unreacted” refers to a norbornene moiety or a derivative thereof and/or tetrazine moiety or derivative thereof which are not bound to another HA chain or moiety.

The amount of unreacted norbornene or a derivative thereof and/or tetrazine or a derivative thereof on the HA chains can be modulated by varying the ratios of the first HA chain to the second HA chain or vice versa during the cross-linking reaction.

Manufacturing Process (Composition with Unmodified HA)

In some embodiments, the present invention is directed to a process of preparing the composition of the invention, the process comprising the steps of: i) mixing a first composition comprising the first (HA) chain (or a derivative thereof) comprising the norbornene moiety (or a derivative thereof) and a second composition comprising the second HA chain (or a derivative thereof) comprising the tetrazine moiety (or a derivative thereof), wherein the first composition, the second composition or both comprises the unmodified HA chain; and (ii) crosslinking the first HA chain to the second HA chain.

In some embodiments, the present invention is directed to a process of preparing the composition of the invention, the process comprising the steps of mixing i) the first (HA) chain (or a derivative thereof) comprising the norbornene moiety (or a derivative thereof), ii) the second HA chain (or a derivative thereof) comprising the tetrazine moiety (or a derivative thereof), and iii) the unmodified HA chain, thereby crosslinking the first HA chain to the second HA chain.

In some embodiments, the present invention is directed to a process of preparing the composition of the invention, the process comprising the steps of mixing i) a first composition comprising the first (HA) chain (or a derivative thereof) comprising the norbornene moiety (or a derivative thereof), ii) a second composition comprising the second HA chain (or a derivative thereof) comprising the tetrazine moiety (or a derivative thereof), and iii) a third composition comprising the unmodified HA chain, thereby crosslinking the first HA chain to the second HA chain.

In some embodiments, the process comprises mixing the unmodified HA with the first HA chain and the second HA chain, prior to the crosslinking of the first HA chain and the second HA chain.

In some embodiments, the composition obtained according to the present process comprises the desired homogeneity and flowability without reduction of the mechanical properties.

In some embodiments, the composition obtained according to the present process is characterized by an improved homogeneity as compared to the property of the same composition obtained by a process where the unmodified HA is mixed with the first HA chain and the second HA chain, after the crosslinking of the first HA chain and the second HA chain.

In some embodiments, the composition obtained according to the present process is characterized by at least one improved mechanical property as compared to the property of the same composition obtained by a process where the unmodified HA is mixed with the first HA chain and the second HA chain, after the crosslinking of the first HA chain and the second HA chain.

In some embodiments, one or more properties selected from elastic modulus, flowability, or phase angle, is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500%. Each possibility represents a separate embodiment of the invention.

Following the attachment of the unsaturated moiety or the tetrazine moiety to the HA chain, the resulting product is purified from unreacted starting materials, as well as well as from other reaction side products. Purification can be performed by any one of the methods known in the art, such as dialysis, precipitation, ultrafiltration or tangential flow filtration.

In some examples, the crosslinking reaction occurs at a usable range of temperature and conditions for forming cross-linked polymers and occurs without the input of external energy. In one embodiment, the crosslinking reaction is heated to increase reaction efficacy.

In some embodiments, the process for preparing the polymer of the invention comprises a spontaneous cross-linking reaction. In some embodiments, by “spontaneous chemical reaction” it is meant to refer to a process that is not assisted by e.g., light, heat, or radicals. In some embodiments, the crosslinking reaction may occur in water, in aqueous buffers or in cell culture media. Non-limiting examples for culture media include, but are not limited to, phosphate buffered saline, Hank's balanced salt solution, Dulbecco's Modified Eagle Medium, and the like. In some embodiments, the cross-linking can occur in organic solvents. Non-limiting examples for organic solvents include, but are not limited to methanol, ethanol, dichloromethane, dimethylformamide, and the like.

In some embodiments, the cross-linking reaction can occur at a wide range of temperatures of at least −80° C., at least −50° C., at least −20° C., at least 0° C., at least 4° C., at least 22° C., at least 37° C., or at least 45° C., and not more than 65° C. In some embodiments, the cross-linking reaction can occur at a wide range of temperatures of (−80)-(−50)° C., (−60)-(−15)° C., (−20)-(−4)° C., (−5)-0° C., (−2)-4° C., 2-8° C., 5-20° C., 15-30° C., 25-40° C., or 35-55° C.

In some embodiments, the steps are performed in-situ, so as to form the cross-linked polymer by mixing the first (HA) chain and the second (HA) chain and the unmodified HA chain. In some embodiments, the preparation process is devoid of a post-processing step such as sieving or homogenization.

In some embodiments of the preparation process, after the cross-linking step, unreacted norbornene or a derivative thereof and/or tetrazine or a derivative thereof may remain attached to the HA chains. As defined herein, the term “unreacted” refers to a norbornene moiety or a derivative thereof and/or tetrazine moiety or derivative thereof which are not bound to another HA chain or moiety.

The amount of unreacted norbornene or a derivative thereof and/or tetrazine or a derivative thereof on the HA chains can be modulated by varying the ratios of the first HA chain to the second HA chain or vice versa during the cross-linking reaction.

### Pharmaceutical Composition

According to another embodiment, the invention provides a pharmaceutical composition comprising the composition of the present invention and a pharmaceutically acceptable carrier.

According to another embodiment, the invention provides a pharmaceutical composition comprising a therapeutically effective amount of the composition of the present invention and a pharmaceutically acceptable carrier. In some embodiments, the pharmaceutical composition or the cosmetic composition comprises (i) the polymer of the invention, or (ii) the polymer of the invention and the unmodified HA chain, wherein the pharmaceutical composition or the cosmetic composition is in a form of a gel (e.g. a hydrogel), further comprising an aqueous solution (e.g. as a cosmetically acceptable or pharmaceutically acceptable carrier).

In some embodiments, there is provided a pharmaceutical composition comprising a therapeutically effective or a cosmetically effective amount of the composition of the present invention and a pharmaceutically acceptable carrier and/or diluent.

In some embodiments, the composition of the present invention further comprises an amino acid. In some embodiments, the amino acid comprises any amino acid, naturally occurring or non-naturally occurring. Non-limiting examples for non-naturally occurring amino acids, include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (NIe), N-methylaminobutyric acid (MeB), 2-naphthylalanine (2Np), aminoheptanoic acid (Ahp), phenylglycine, β-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-,3-, and 4-(aminomethyl)-benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAI). In some embodiments, the amino acid is an oligomer, or a dimer of amino acids linked by a peptide bond. In some embodiments, the oligomer is a trimer, a tetramer, a pentamer, a hexamer, a heptamer, an octamer, a nonamer, a decamer, or, in some embodiments, is a polymer having more than 11 amino acids bound to one another by peptide bonds. In some embodiments, amino acids are included in the composition form a peptide, a polypeptide or a protein. In some embodiments, the peptide, polypeptide or the protein included in a composition of the present invention, is in the form selected from, without being limited thereto, native, denaturated, neutralized, digested, cross-linked, un-folded, reduced, oxidized, or inactivated form.

In some embodiments, the composition of the present invention further comprises a mineral or a plurality of minerals. Non-limiting examples for a mineral include, but are not limited to: Potassium, Chloride, Sodium, Calcium, Phosphorus, Magnesium, Iron, Zinc, Manganese, Copper, Iodine, Chromium, Molybdenum, Selenium or Cobalt.

In some embodiments, the composition of the present invention further comprises a vitamin. Non-limiting examples of vitamins include, but are not limited to: Vitamin A (Retinol, retinal and four carotenoids including beta carotene), Vitamin B₁ (Thiamine), Vitamin B₂ (Riboflavin), Vitamin B₃ (Niacin, niacinamide, Nicotinamide riboside), Vitamin B₅ (Pantothenic acid), Vitamin B₆ (Pyridoxin, pyridoxamine, pyridoxal), Vitamin B₇ (Biotin), Vitamin B₉ (Folates), Vitamin B₁₂ (Cyanocobalamin, hydroxocobalamin, methylcobalamin, adenosylcobalamin), Vitamin C (Ascorbic acid), Vitamin D (Cholecalciferol (D₃), Ergocalciferol (D₂)), Vitamin E (Tocopherols, tocotrienols), or Vitamin K (Phylloquinone, menaquinones).

In some embodiments, the composition of the present invention further comprises an anesthetic agent. As used herein, the term “anesthetic” refers to any molecule or substance which prevents pain such as during surgery, or completely blocks any feeling. In one embodiment, the anesthetic agent is general anesthetic agent. In one embodiment, the anesthetic agent is a local anesthetic agent. In some embodiment, a local anesthetic agent induces a reversible loss of sensation limited to a certain region of the body while maintaining consciousness.

Non-limiting examples for anesthetic agents include, but are not limited to articaine, procaine, amethocaine, lidocaine, bupivacaine, levobupivacaine, ropivacaine, mepivacaine, dibucaine and cocaine. In some embodiments, the composition of the present invention comprises 0.01-0.1% (w/w), 0.05-0.15% (w/w), 0.1-0.3% (w/w), 0.2-0.5% (w/w), 0.4-0.7% (w/w), 0.6-0.85% (w/w), 0.8-1.25% (w/w), 1-1.5% (w/w), 1.4-2% (w/w), 1.75-3% (w/w), 2.5-3.75% (w/w), 3.5-4.5% (w/w), or 4.25-5.25% (w/w) of an anesthetic agent. In some embodiments, the composition of the present comprises at most 0.01% (w/w), at most 0.05% (w/w), at most 0.75% (w/w), at most 1% (w/w), at most 1.5% (w/w), at most 2% (w/w), at most 3% (w/w), at most 4% (w/w), or at most 5.5% (w/w) of an anesthetic agent. Each possibility represents a separate embodiment of the present invention.

In another embodiment, the pharmaceutical composition of the invention may be formulated in the form of a pharmaceutically acceptable salt of the polymer of the present invention. In another embodiment, pharmaceutically acceptable salts include salts derived from non-toxic inorganic or organic acids such as hydrochloric, phosphoric, acetic, oxalic, tartaric acids, and the like. In some embodiments, the salts are formed with free carboxyl groups such as salts derived from non-toxic inorganic or organic bases such as sodium, potassium, ammonium, calcium, ferric hydroxides, isopropylamine, triethylamine, 2-ethylamino ethanol, histidine, procaine, and the like.

As used herein, the term “carrier” refers to a diluent, adjuvant, excipient, or vehicle administered with the disclosed compound. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.9% by weight of the pharmaceutical compositions presented herein.

As used herein, the term “pharmaceutically acceptable” means suitable for administration to a subject, e.g., a human. For example, the term “pharmaceutically acceptable” can mean approved by a regulatory agency of the Federal or a state government or listed in the U.S. Pharmacopeia or other generally recognized pharmacopeia for use in animals, and more particularly in humans.

In another embodiment, the composition of the invention takes the form of solutions, suspensions, emulsions, tablets, powders, gels, foams, pastes, sustained-release formulations and the like. Examples of suitable pharmaceutical carriers are described in: Remington's Pharmaceutical Sciences” by E.W. Martin, the contents of which are hereby incorporated by reference herein. Such compositions will contain a therapeutically effective amount of the polymer of the invention, preferably in a substantially purified form, together with a suitable amount of carrier so as to provide the form for proper administration to the subject.

According to an embodiment of the invention, a pharmaceutical composition contains 0.1-95% of the polymer(s) and/or composition of the present invention. According to another embodiment of the invention, a pharmaceutical composition contains 1-70% of the polymer and/or composition. According to another embodiment of the invention, the composition or formulation to be administered may contain a quantity of polymers and/or compositions, according to embodiments of the invention in an amount effective to treat the condition or disease of the subject being treated.

In some embodiments, the pharmaceutical composition is for use in the treatment of a medical, cosmetic and/or cosmeceutical condition.

In some embodiments, the pharmaceutical composition as described herein is a topical composition. In some embodiments, the pharmaceutical composition is an injectable composition. In some embodiments, the pharmaceutical composition is for a systemic use.

In some embodiments, the pharmaceutical composition is any of an emulsion, a liquid solution, a gel, a paste, a suspension, a dispersion, an ointment, a cream or a foam.

The pharmaceutical compositions of this invention can be administered to mammals (e.g., humans, rodents, etc.) in any suitable way including, e.g., orally, parenterally, intracisternally, intraperitoneally, topically, etc. The parenteral administration includes intravenous, intramuscular, intraperitoneal, intrasternal, subcutaneous and intraarticular injection/infusion.

The dose will vary depending on the subject and upon the particular route of administration used. Commercially available assays may be employed to determine optimal dose ranges and/or schedules for administration. Effective doses may be extrapolated from dose-response curves obtained from animal models.

An embodiment of the invention relates to the polymer and/or the composition of the present invention, presented in unit dosage form and prepared by any of the methods well known in the art of pharmacy. In an embodiment of the invention, the unit dosage form is in the form of a tablet, capsule, lozenge, ampoule, vial or pre-filled syringe. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

According to one embodiment, the composition of the present invention is administered in the form of a pharmaceutical composition comprising at least one of the active components of this invention (the polymer) together with a pharmaceutically acceptable carrier or diluent. In another embodiment, the composition of this invention can be administered either individually or together in any conventional transdermal dosage form.

As used herein, the terms “administering”, “administration”, and like terms refer to any method which, in sound medical practice, delivers a composition containing an active agent to a subject in such a manner as to provide a therapeutic effect.

Depending on the location of the tissue of interest, the polymer and/or the composition of the present invention can be administered in any manner suitable for the provision of the polymer and/or the composition to the tissue of interest. Thus, for example, a composition containing the polymer of the present invention can be introduced, for example, injected into the tissue of interest which will distribute the polymer in the tissue.

In some embodiments, the pharmaceutical composition comprising the polymer is administered via ophthalmic, transdermal, intradermal, subcutaneous, intramuscular, or intraperitoneal routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of polymers administered by other routes can be lower than when administered via parenteral injection, by using appropriate formulations it is envisaged that it will be possible to administer the composition of the invention via transdermal, oral, rectal, vaginal, topical, nasal, inhalation and ocular modes of treatment.

For topical application, the polymer and/or the composition of the present invention can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

According to some embodiments, the polymer and/or the composition of the present invention, can be delivered in a controlled release system. In yet another embodiment, a controlled release system can be placed in proximity to a therapeutic target, thus requiring only a fraction of the systemic dose.

In one embodiment, it will be appreciated that the polymer and/or the composition of the present invention can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the polymer and/or the composition is administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the polymer is the amount of the polymer necessary for the in vivo measurable expected biological or therapeutic effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated or the defect to be corrected, the condition of the individual patient, the site of delivery, the method of administration and other factors known to practitioners. Examples of techniques and protocols can be found in Remington: The Science and Practice of Pharmacy, 21^{st} Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

Pharmaceutical compositions containing the presently described polymer as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

In one embodiment, composition including the preparation of the present invention formulated in a compatible pharmaceutical carrier is prepared, placed in an appropriate container, and labeled for treatment of an indicated condition.

In one embodiment, composition of the present invention is presented in a pack or dispenser device, such as an FDA approved kit, which contains, one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Methods of Use

In some embodiments, there present invention is directed to a method for filling or volumizing a tissue in a subject in need thereof, the method comprising administering to the subject a pharmaceutical composition comprising a therapeutically effective amount of the composition of the invention.

In some embodiments, there present invention is directed to a method for filling or volumizing a tissue in a subject in need thereof, the method comprising administering to the subject a pharmaceutical composition comprising a therapeutically effective amount of a polymer of the invention.

In some embodiments, a filling or a volumizing method, as defined herein, refers to the injection of a soft filling material into a skin tissue. In some embodiments, the filling is filling wrinkles, such as facial wrinkles. In some embodiments, filling is restoring a smooth appearance or texture of the skin.

In some embodiments, the method is directed to injection of a polymer or a composition of the present invention into a skin tissue. In some embodiments, the method is directed to injection of a polymer or a composition of the present invention into a gingival tissue. In some embodiments, the method is directed to injection of a polymer or a composition of the present invention into a cartilage tissue. In some embodiments, the method is directed to injection of a polymer or a composition of the present invention into an ophthalmic tissue.

According to some embodiments, injection can be performed according to any method and using any injecting device known in the art. Non-limiting examples of injecting devices include, but are not limited to the use of syringes, microsyringes, needleless devices, microneedling, needles, cannula and catheters. Non-limiting examples of needle gauges include but are not limited to, 18G, 19G, 20G, 21G, 22G, 23G, 24G, 25G, 26G, 27G, 28G, 29G, 30G, 31G, 32G, 33G, or 34G. In some embodiments, the injection is via an injecting device driven by a human power.

In some embodiments, the method of the present invention is directed to treating arthritis in a subject in need thereof. Non-limiting examples for arthritis include, but are not limited to, acute infectious arthritis, calcium pyrophosphate arthritis, arthritis of the temporomandibular joint (TMJ), reactive arthritis, psoriatic arthritis, chronic infectious arthritis, juvenile idiopathic arthritis (JIA), rheumatoid arthritis (RA), or prosthetic joint infectious arthritis.

In one embodiment, the method of the present invention is directed to treating a subject afflicted with osteoarthritis.

In some embodiments, the method of the present invention is directed to preventing or treating an ophthalmic tissue damage in a subject prior to or after a surgical procedure.

In some embodiments, the method is directed to topical application of a polymer or a composition of the present invention. In some embodiments, the polymer of the present invention is applied on a skin tissue. In some embodiments, the method is directed to promoting/enhancing wound healing, in a subject in need thereof. In some embodiments, the method is directed to promoting/enhancing wound closure, in a subject in need thereof.

In one embodiment, the polymer of the present invention is provided to the subject per se. In one embodiment, one or more of the polymers of the present invention are provided to the subject per se. In one embodiment, the compositions of the present invention are provided to the subject per se. In one embodiment, the polymer of the present invention is provided to the subject as part of a pharmaceutical composition where it is mixed with a pharmaceutically acceptable carrier. In one embodiment, one or more of the polymers of the present invention are provided to the subject as part of a pharmaceutical composition where they are mixed with a pharmaceutically acceptable carrier. In one embodiment, the composition of the present invention is provided to the subject as part of a pharmaceutical composition where they are mixed with a pharmaceutically acceptable carrier.

The term “subject” as used herein refers to an animal, more particularly to non-human mammals and human organism. Non-human animal subjects may also include prenatal forms of animals, such as, e.g., embryos or fetuses. Non-limiting examples of non-human animals include but are not limited to: horse, cow, camel, goat, sheep, dog, cat, non-human primate, mouse, rat, rabbit, hamster, guinea pig, or pig. In one embodiment, the subject is a human. Human subjects may also include fetuses. In one embodiment, a subject in need thereof is a subject afflicted with and/or at risk of being afflicted with a condition associated with arthritis. In some embodiments, a subject in need thereof is a subject afflicted by reduction of tissue volume. In some embodiments, reduction of tissue volume is referred to as “devolumization”. In some embodiments, tissue devolumization comprises fat loss, water loss, moisture loss, extracellular matrix degradation, collagen loss, or others. In some embodiments, devolumization induces skin sagging and descent. In some embodiments, a subject suffers from burns. In some embodiments, a subject in need of a method for wound closure suffers from leakage of body fluids, such as bleeding. As used herein, the terms “treatment” or “treating” of a disease, disorder, or condition encompasses alleviation of at least one symptom thereof, a reduction in the severity thereof, or inhibition of the progression thereof. Treatment does not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described polymer or composition comprising the polymer prior to the induction or onset of the disease/disorder process. In any case, the term prophylaxis can be applied to encompass prevention. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

### The Kit

According to some embodiments, the present invention provides a kit comprising a first HA chain or a derivative thereof as described hereinabove and a second HA chain or a derivative thereof as described hereinabove, wherein the first HA chain, the second HA chain or both comprise unmodified HA chain characterized by an average molecular weight (MW) between 300 kiloDaltons (kDa) and 4,000 kDa.

According to some embodiments, the present invention provides a kit comprising a first HA chain or a derivative thereof coupled to an unsaturated moiety or a derivative thereof and a second HA chain or a derivative thereof coupled to a tetrazine moiety or a derivative thereof, wherein the first HA chain, the second HA chain or both comprise unmodified HA chain characterized by an average molecular weight (MW) between 300 kiloDaltons (kDa) and 4,000 kDa. The terms “HA chain or a derivative thereof”, “unsaturated moiety or a derivative thereof” and “tetrazine moiety or a derivative thereof” are as defined herein above.

In some embodiments, the kit comprises an injecting device. In some embodiments, the kit comprises a device for injecting in or through the skin or a device for microperforation of the skin, designated to the administration of the dose.

According to some embodiments, the present invention provides a kit comprising the polymer of the invention. In some embodiments, the kit further comprises the unmodified HA chain, and/or a carrier, as described herein. In some embodiments, the polymer of the invention and the unmodified HA chain are stored in separate containers. In some embodiments, the polymer of the invention and the unmodified HA chain are in a form of a gel (e.g. a hydrogel), further comprising an aqueous solution (e.g. as a cosmetically acceptable or pharmaceutically acceptable carrier). In some embodiments, the kit comprises any of the compositions disclosed herein.

In some embodiments, the kit comprises a first hyaluronic acid (HA) chain or a derivative thereof and a second HA chain or a derivative thereof, wherein said first HA chain and said second HA chain are crosslinked via one or more linkers, wherein said one or more linkers comprise norbornene moiety or a derivative thereof coupled to a tetrazine moiety or a derivative thereof, and wherein said polymer is characterized by a crosslinking degree of 0.6 to 4%, which is predetermined by the modification degree of the first hyaluronic acid (HA) chain and of the second HA chain, as determined by 1H NMR (as described in greater detail in WO2020003298).

In some embodiments, an injecting device is as disclosed above. In one embodiment, an injecting device is disposable. In some embodiments, the injecting device is suitable for an intraepidermal and/or intradermal and/or subcutaneous injection. In some embodiments, the injecting device is suitable for mesotherapy technique. As used herein, the term “mesotherapy” refers to a non-surgical cosmetic medicine treatment.

In some embodiments, the kit further comprises an amino acid as disclosed above.

In some embodiments, the kit further comprises a mineral as disclosed above.

In some embodiments, the kit further comprises a vitamin as disclosed above.

In some embodiments, the kit further comprises an anesthetic as disclosed above.

In some embodiments, the kit further comprises a pharmaceutically acceptable carrier as disclosed above.

In some embodiments, the kit comprises any of the compositions disclosed herein.

In some embodiments, the packaging is scored so as to allow the first HA chain or a derivative thereof coupled to a norbornene moiety or a derivative thereof, the second HA chain or a derivative thereof coupled to a tetrazine moiety or a derivative thereof, and optionally the unmodified HA chain characterized by an average molecular weight (MW) between 300 kiloDaltons (kDa) and 4,000 kDa the amino acid, the vitamin, the mineral, the anesthetic, or any combination thereof to be sampled.

In one embodiment, a packaging is in the form of an ampoule, a bottle or a capsule. In one embodiment, a capsule is a soft capsule.

In some embodiments, the components of the kit disclosed above are sterile. As used herein, the term “sterile” refers to a state of being free from biological contaminants. Any method of sterilization is applicable and would be apparent to one of ordinary skill in the art.

In some embodiments, the kit is utilized by mixing a first HA chain or a derivative thereof coupled to a norbornene moiety or a derivative thereof and a second HA chain or a derivative thereof coupled to a tetrazine moiety or a derivative thereof, and applying the composition formed by mixing a first HA chain or a derivative thereof coupled to a norbornene moiety or a derivative thereof and a second HA chain or a derivative thereof coupled to a tetrazine moiety or a derivative thereof to a filling or volumizing method.

In some embodiments, the kit is utilized by further mixing the composition formed by mixing a first HA chain or a derivative thereof coupled to a norbornene moiety or a derivative thereof and a second HA chain or a derivative thereof coupled to a tetrazine moiety or a derivative thereof, and a unmodified HA chain, and applying the composition formed by mixing the first HA chain or a derivative thereof coupled to a norbornene moiety or a derivative thereof and the second HA chain or a derivative thereof coupled to a tetrazine moiety or a derivative thereof, and the unmodified HA chain to a filling or volumizing method.

In some embodiments, the kit is utilized by further mixing the composition formed by mixing a first HA chain or a derivative thereof coupled to a norbornene moiety or a derivative thereof and a second HA chain or a derivative thereof coupled to a tetrazine moiety or a derivative thereof, and an unmodified HA chain, an amino acid, a vitamin, a mineral, an anesthetic, or any combination thereof and applying the composition formed by mixing the first HA chain or a derivative thereof coupled to a norbornene moiety or a derivative thereof and the second HA chain or a derivative thereof coupled to a tetrazine moiety or a derivative thereof, and the unmodified HA chain, the amino acid, the vitamin, the mineral, the anesthetic, or any combination thereof to a filling or volumizing method.

In some embodiments, the kit comprises a first composition comprising the first HA chain or the derivative thereof; and a second comprising the second HA chain or the derivative thereof; wherein a concertation of the first HA chain within the first composition and a concertation of the second HA chain and within the second composition is independently between 1 and 20 mg/g, between 2 and 20 mg/g, between 2 and 10 mg/g, between 2 and 9 mg/g, between 2 and 8 mg/g, between 3 and 20 mg/g, between 4 and 20 mg/g, between 4 and 10 mg/g, between 4 and 8 mg/g, including any range between.

In some embodiments, the kit comprises instructions for mixing the first HA chain or a derivative thereof coupled to a norbornene moiety or a derivative thereof, and the second HA chain or a derivative thereof coupled to a tetrazine moiety or a derivative thereof in a ratio of about 3:1, about 3:2, about 2:1, about 1:1, about 1:2, including any range between; wherein norbornene moiety or a derivative thereof and the tetrazine moiety or a derivative thereof are as described herein.

In some embodiments, the kit comprises instructions for mixing a first HA chain or a derivative thereof coupled to a norbornene moiety or a derivative thereof, a second HA chain or a derivative thereof coupled to a tetrazine moiety or a derivative thereof and an unmodified HA chain.

In some embodiments, the kit is utilized by mixing a first HA chain or a derivative thereof coupled to a norbornene moiety or a derivative thereof, a second HA chain or a derivative thereof coupled to a tetrazine moiety or a derivative thereof and an unmodified HA chain, wherein mixing comprises introducing the components in the injecting device.

In some embodiments, the kit comprises instructions for mixing a first HA chain or a derivative thereof coupled to a norbornene moiety or a derivative thereof and a second HA chain or a derivative thereof coupled to a tetrazine moiety or a derivative thereof and an unmodified HA chain, wherein mixing is performed in the injecting device.

In some embodiments of the subject kit, the composition formed by mixing a first HA chain or a derivative thereof coupled to an unsaturated moiety (e.g. norbornene moiety) or a derivative thereof and a second HA chain or a derivative thereof coupled to a tetrazine moiety or a derivative thereof and an unmodified HA is characterized by a phase angle (δ) between 0.1° and 10°, 0.1° and 0.5°, 0.1° and 0.9°, 0.1° and 1°, 0.1° and 1.5°, 0.1° and 4.5°, 0.1° and 7.5°, 0.1° and 8.5°, 0.1 and 9.5°, 0.5° and 10°, 0.5° and 0.9°, 0.5° and 1°, 0.5° and 1.5°, 0.5° and 4.5°, 0.5° and 7.5°, 0.5° and 8.5°, 0.5 and 9.5°, 0.9°, and 10°, 0.9° and 0.5°, 0.9° and 1°, 0.9° and 1.5°, 0.9° and 4.5°, 0.9° and 7.5°, or between 0.9° and 8.5°, including any range therebetween. Each possibility represents a separate embodiment of the present invention.

In some embodiments of the subject kit, the composition formed by mixing a first HA chain or a derivative thereof coupled to a norbornene moiety or a derivative thereof and a second HA chain or a derivative thereof coupled to a tetrazine moiety or a derivative thereof and an unmodified HA chain, is characterized by an elastic modulus between 40 Pa and 400 Pa, 45 Pa and 400 Pa, 50 Pa and 400 Pa, 70 Pa and 400 Pa, 80 Pa and 400 Pa, 40 Pa and 400 Pa, 100 Pa and 400 Pa, 45 Pa and 350 Pa, 50 Pa and 350 Pa, 70 Pa and 350 Pa, 80 Pa and 350 Pa, 95 Pa and 350 Pa, 150 Pa and 350 Pa, 48 Pa and 250 Pa, 55 Pa and 250 Pa, 70 Pa and 250 Pa, 80 Pa and 250 Pa, 90 Pa and 250 Pa, or between 100 Pa and 250 Pa, including any range therebetween. Each possibility represents a separate embodiment of the present invention.

In some embodiments of the subject kit, the composition formed by mixing a first HA chain or a derivative thereof coupled to a norbornene moiety or a derivative thereof and a second HA chain or a derivative thereof coupled to a tetrazine moiety or a derivative thereof and an unmodified HA chain, is characterized by an average extrusion force between 5 N and 30 N, 7 N and 20 N, 8 N and 20 N, 10 N and 20 N, 5 N and 18 N, 7 N and 18 N, 8 N and 18 N, 10 N and 18 N, 5 N and 15 N, 7 N and 15 N, 8 N and 15 N, or between 10 N and 15 N, including any range therebetween. Each possibility represents a separate embodiment of the present invention.

In some embodiments of the subject kit, the composition formed by mixing a first HA chain or a derivative thereof coupled to a norbornene moiety or a derivative thereof and a second HA chain or a derivative thereof coupled to a tetrazine moiety or a derivative thereof and an unmodified HA chain, is characterized by a maximum extrusion force between 5 N and 30 N, 7 N and 20 N, 8 N and 20 N, 10 N and 20 N, 5 N and 18 N, 7 N and 18 N, 8 N and 18 N, 10 N and 18 N, 5 N and 15 N, 7 N and 15 N, 8 N and 15 N, or between 10 N and 15 N, including any range therebetween. Each possibility represents a separate embodiment of the present invention.

In some embodiments of the subject kit, the composition formed by mixing a first HA chain or a derivative thereof coupled to a norbornene moiety or a derivative thereof and a second HA chain or a derivative thereof coupled to a tetrazine moiety or a derivative thereof and an unmodified HA chain, is characterized by an extrusion profile devoid of peaks higher than 2 N, higher than 1.9 N, higher than 1.8 N, higher than 1.7 N, higher than 1.5 N, or higher than 1 N, including any value therebetween. Each possibility represents a separate embodiment of the present invention.

In some embodiments, the components of the kit are packaged within a container.

In some embodiments, the container is made of a material selected from the group consisting of thin-walled film or plastic (transparent or opaque), paperboard-based, foil, rigid plastic, metal (e.g., aluminum), glass, etc.

In some embodiments, the content of the kit is packaged, as described below, to allow for storage of the components until they are needed.

In some embodiments, some or all components of the kit may be packaged in suitable packaging to maintain sterility.

In some embodiments, the packaging has a cap which allows hermetic sealing during storage and which can be pierced by a needle or cannula at the time of use.

In some embodiments, the components of the kit are stored in separate containers within the main kit containment element e.g., box or analogous structure, may or may not be an airtight container, e.g., to further preserve the sterility of some or all of the components of the kit.

In some embodiments, the dosage amount of the first HA chain or a derivative thereof coupled to a norbornene moiety or a derivative thereof, the second HA chain or a derivative thereof coupled to a tetrazine moiety or a derivative thereof, the unmodified HA chain, the amino acid, the vitamin, the mineral, or the anesthetic provided in a kit may be sufficient for a single application or for multiple applications.

In some embodiments, the kit may have multiple dosage amounts of the first HA chain or a derivative thereof coupled to a norbornene moiety or a derivative thereof, the second HA chain or a derivative thereof coupled to a tetrazine moiety or a derivative thereof, the unmodified HA chain, the amino acid, the vitamin, the mineral, or the anesthetic packaged in a single container, e.g., a single tube, bottle, vial, Eppendorf and the like.

In some embodiments, the kit may have multiple dosage amounts of the first HA chain or a derivative thereof coupled to a norbornene moiety or a derivative thereof, the second HA chain or a derivative thereof coupled to a tetrazine moiety or a derivative thereof, the unmodified HA chain, the amino acid, the vitamin, the mineral, or the anesthetic individually packaged such that certain kits may have more than one container of first HA chain or a derivative thereof coupled to a norbornene moiety or a derivative thereof, the second HA chain or a derivative thereof coupled to a tetrazine moiety or a derivative thereof, the unmodified HA chain, the amino acid, the vitamin, the mineral, or the anesthetic.

In some embodiments, multiple dosage amounts of the first HA chain or a derivative thereof coupled to a norbornene moiety or a derivative thereof, the second HA chain or a derivative thereof coupled to a tetrazine moiety or a derivative thereof, the unmodified HA chain, the amino acid, the vitamin, the mineral, or the anesthetic may be packed in single separate containers.

In some embodiments, the kit contains instructions for preparing the composition used therein and for how to practice the methods of the invention.

In some embodiments, the instructions may be recorded on a suitable recording medium or substrate. For example, the instructions may be printed on a substrate, such as paper or plastic, etc.

In some embodiments, the instructions may be present in the kit as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In another aspect, there is provided a process for manufacturing the polymer of the invention, or the composition comprising the polymer and the unmodified HA, as described herein.

In some embodiments, the method of manufacturing the polymer of the invention comprises mixing the first HA chain or a derivative thereof coupled to a norbornene moiety or a derivative thereof, and the second HA chain or a derivative thereof coupled to a tetrazine moiety or a derivative thereof at a molar ratio or at a weight ratio (between the first HA chain and the second HA chain) of about 3:1, about 3:2, about 2:1, about 1:1, about 1:2, including any range between. In some embodiments, the first HA chain and the second HA chain are each independently in a form of an aqueous composition (e.g. a solution, dispersion or a suspension). In some embodiments, a concentration of the first HA chain or of the second HA chain within the aqueous composition is as described hereinabove (.e.g between 1 and 20 mg/g).

In some embodiments, the method comprises mixing a predetermined amount of a first composition with the second composition under appropriate conditions.

In some embodiments, the predetermined amount comprises the concentration of the first HA chain or of the second HA chain as described herein, and a molar ratio or a weight ratio between the first HA chain and the second HA chain of about 3:1, about 3:2, about 2:1, about 1:1, about 1:2, about 1:3, between 1.5:1 and 1:1.5 including any range between.

In some embodiments, the appropriate conditions comprise an ambient atmosphere and a temperature between 0 and 60° C., between 0 and 30° C., between 10 and 30° C., between 10 and 60° C., between 30 and 60° C., including any range between.

In some embodiments, the appropriate conditions a reaction time (under mixing or stirring) between 1 second and 2 weeks, including any range between. In some embodiments, the appropriate conditions further comprise aging (e.g. at a temperature between 0 and 60° C. and without stirring or mixing) between 1 and 30 days, between 1 and 15 days, including any range between.

In some embodiments, the method of manufacturing the composition comprising the polymer and the unmodified HA comprises mixing a predetermined amount of the first composition with the second composition under appropriate conditions at a molar ratio or at a weight ratio between the first HA chain and the second HA chain of about 3:1, about 3:2, about 2:1, about 1:1, about 1:2, about 1:3, between 1.5:1 and 1:1.5 including any range between, wherein the appropriate conditions, mixing, the first composition and the second composition are as described herein, and wherein the first composition and/or the second composition further comprise the unmodified HA.

In some embodiments, a concentration of the unmodified HA within the first composition and/or the second composition is between 5% and 60%, between 5% and 30%, between 10% and 60%, between 10% and 30%, between 5% and 20%, between 5% and 40%, between 10% and 20%, between 20% and 60%, between 20% and 40%, between 40% and 60%, including any range between.

### Definitions

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range; e.g., “21-100”, is stated herein, it implies that the group, in this case the alkyl group, may contain 21 carbon atoms, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” describes a —C(═O)—OR′ group, where R′ is hydrogen, alkyl, cycloalkyl, alkenyl, aryl, heteroaryl (bonded through a ring carbon) or heteroalicyclic (bonded through a ring carbon), as defined herein; or wherein R′ is absent, e.g. carboxylate anion and/or carboxylate salt.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂-R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a —C═N group.

As used herein, the term “azide” refers to a —N₃ group.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

### General

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a” “an” and “at least one” are used interchangeably in this application.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Materials and Methods

Preparation of the second HA chain (e.g. HA-tetrazine) and of the first HA chain (e.g. HA-norbornene). Exemplary synthetic procedures are as described in greater detail in WO2020003298.

Preparation of the crosslinked polymer via cross-linking the first HA chain (e.g. HA-norbornene) and the second HA chain (e.g. HA-tetrazine)

In an exemplary procedure, HA-tetrazine and HA-norbornene were separately dissolved in a phosphate buffer solution at the pH value ranging from 7.0 to 8.0, so as to obtain aqueous solutions HA-tetrazine and HA-norbornene, respectively with a concentration independently ranging between 1 and 20 mg/g, or between 1 and 10 mg/g. Substantially the same volume of the two solutions were mixed and left at 37° C. for 24 hours and then further at room temperature until the rheological gel parameters were stable.

The crosslinking degree of the obtained gel was the mean of the modification degrees of the HA-tetrazine and HA-norbornene used.

### Example 1

### Determination of the sHA Content

Exemplary compositions comprising crosslinked polymers of the invention have been tested for the sHA content of each composition. Furthermore, the sHA content of the commercially available dermal fillers has been determined, as described hereinbelow. In brief, the sHA content has been determined according to HPLC-based method, and by carbazole assay.

Additionally, the extrusion force for each tested composition has been determined. The results of these tests are summarized in Tables 1 and 2.

### Extrusion Force Measurement

Extrusion force was measured using a Mecmesin traction bench instrument. The data was acquired by extrusion of gel from 1 ml syringe equipped with 27G ½″ or 29 ½″ or 30G ½″ needles at a standard rate of 12.5 mm/min. For the tested gels, no sieving or fragmentation was performed.

### Determination of the sHA Content (HPLC-Based Method)

Analytical data for each of the HA-containing samples reported the (μg) mass of extracted soluble HA detected in the hydrogel samples, its molecular weight (Mw), polydispersity (i.e. p/d) (Mw/Mn), and the Rz-value (nm). In addition, the soluble HA (sHA) fraction was determined for multiple molecular weight (Mw) limits of <50 kDa, <100 kDa and <250 kDa. The sHA content has been determined quantified by HPLC, by utilizing validated HPLC methods.

These results are detailed in Table 2 where they are represented as the characterization profile for % sHA and corrected % sHA for the Mw limits of <50 kDa, <100 kDa and <250 kDa. % sHA fractions are normalized according to HA content (mg/g) for valid comparable Mw limit results.

High Pressure Liquid Chromatography, Size Exclusion Chromatography, Multi-Angle Light Scattering and Refractive Index Detectors (HPLC-SEC-MALS-RI) were interfaced to detect the sHA released after dilution and separation via filtration. Analytical data for sHA relied on a well understood method of using a known concentration (mg/mL) of sample diluted in a known quantity (mL) of a 0.1 μm filtered 150 mM Sodium Nitrate solution (Mobile Phase). The diluted mixture consisted of modified (mHA) and soluble (sHA) hyaluronate—The Sample Composition. The highly networked and larger size mHA portion is separated from the sHA portion via the use of a 0.45 um filter. The resulting filtered material, sHA, is used to furnish a sample suitable for HPLC-SEC-MALS-RI analysis.

Agilent Technologies HPLC interfaced Wyatt Technology's Dawn 18-angle MALS and Optilab RI Detector. The Size Exclusion Chromatography (SEC) was two (2) Shodex LB-806M columns.

Absolute molecular weights for sHA (Molar Mass)—HPLC-SEC-MALS-RI determines the absolute molecular weights for sHA, the unique molar and sample masses (g) of molecules, molar mass (g/mol).

Mobile Phase—A Sodium Nitrate solution is used for the mobile phase.

Change in refractive index with concentration of HA (dn/dc)—The quantitative specificity of HPLC-SEC-MALS-RI methods rely on the use of reliable dn/dc-values. These values establish the change in the index of refraction (dn) for an analyte, such as HA, with respect to changes in its concentration (dc) within a specified solvent at a certain temperature. The dn/dc value 0.165 mL/g for Sodium Nitrate mobile phase was used to quantify and characterize sHA using the Wyatt ASTRA® software.

The analytical results presented herein were based on two HPLC-SEC-MALS-RI system injections of the samples. Injection volume (μL) variations were automatically accounted for within the Astra software collection cycle(s).

### Determination of the sHA Content (Carbazole Assay)

The gels (about 200 mg) were weighted into a 15 mL falcon tube. An excess of 7 mL of phosphate buffered saline (PBS) was added to each tube. The resulting mixtures were homogenized by vortex and incubated of 7 hours at 37° C., while the samples were gently rotated. After incubation, the samples were filtered through 0.45 μm Nylon filter to separate the non-soluble gel from the soluble HA fraction in the filtrates. The filtrate samples were diluted with PBS in order to fit in the assay calibration range and then the HA concentration was determined using a Carbazole Assay (adapted from the Assay described in the Sodium Hyaluronate monograph in the European Pharmacopoeia). The HA concentrations in the filtrate were converted into the corresponding concentration of soluble HA in the initial gel. Experiments were conducted in triplicate.

As shown in Table 1, the extrusion force values of the exemplary compositions of the invention are within the acceptable range.

TABLE 1
sHA content (as determined by carbazole assay) and extrusion force values

| | | | | | | | Extrusion | |
|---|---|---|---|---|---|---|---|---|
| | Product | HA | | Crosslinking | G′ | δ | force | % |
| Ent. | name | (mg/gr) | Linker | degree | (Pa) | (°) | (N) | sHA |
| 1 | Restylane | 20 | BDDE | 1% | 705 | 9.8 | 12.6 | 14.7% |
| | Lidocaine | mg/gr | | | | | | |
| 2 | | 6 | IIA/IIIB | 0.45% | 16 | 14.5 | 14.4 | 14.3% |
| | | mg/gr | | | | | | |
| 3 | | 6 | IIA/IIIB | 0.8% | 33 | 3.4 | 15.7 | 2.7% |
| | | mg/gr | | | | | | |
| 4 | | 6 | IIA/IIIB | 1.0% | 50 | 2.2 | 18.2 | 3.5% |
| | | mg/gr | | | | | | |
| 5 | | 6 | IIA/IIIB | 1.4% | 107 | 1.9 | 21.5 | 1.3% |
| | | mg/gr | | | | | | |
| 6 | | 4 | IIA/IIIB | 0.8% | 16 | 5.5 | 14.7 | 5.2% |
| | | mg/gr | | | | | | |
| 7 | | 4 | IIA/IIIB | 1.0% | 23 | 3.4 | 18.8 | 4.2% |
| | | mg/gr | | | | | | |
| 8 | | 10 | IIA/IIIB | 0.8% | 56 | 3.3 | 21.1 | 2.9% |
| | | mg/gr | | | | | | |
| 9 | | 10 | IIA/IIIB | 1.0% | 83 | 2.2 | 23.9 | 2.75% |
| | | mg/gr | | | | | | |
| 10 | | 8 | IIA/IIIB | about 3% | 212 | 0.8 | 26.1 | 0.6% |
| | | mg/gr | | | | | | |

Entry 1 represents a commercial dermal filler with a crosslinking degree of 1%, and characterized by a fraction of sHA higher than 10% (14.7%).

Entries 2-5 show the effect of the crosslinking degree on the sHA content of exemplary composition of the invention. Crosslinking at 0.45% is not enough to ensure a soluble fraction inferior to 10% (Entry 2). Crosslinking degrees of 0.8%, 1.0% and 1.4% ensure a soluble fraction lower than 10% (Entries 3 to 5).

Entries 3, 4 and 6-9 show the effect of the HA concentration on the sHA content of exemplary composition of the invention at a crosslinking degree of 0.8% (Entries 3, 6, 8) and 1.0% (Entries 4, 7, 9). In the concentration range studied (4-8 mg/g) all the tested exemplary composition of the invention comply with a soluble HA fraction lower than 10%.

Entry 10 shows the effect of a higher crosslinking degree of about 3%, with a very low sHA fraction (0.6%). In this composition the measured extrusion force was in the acceptable range (not exceeding 30 N), such that the composition is considered extrudable.

TABLE 2
sHA content (as determined by HPLC)

| | | | | | | % sHA | % sHA | % sHA |
|---|---|---|---|---|---|---|---|---|
| | Product | Total | | Crosslinking | % | (MW <50 | (MW <100 | MW <250 |
| Ent. | name | HA | Linker | degree | sHA | kDa) | kDa) | kDa |
| 1 | Restylane | 20 | BDDE | 1% | 16.40 | 1.70 | 5.70 | 12.80 |
| | Lidocaine | mg/gr | | | | | | |
| 2 | Juvederm | 20 | BDDE | 6% | 26.70 | 9.70 | 19.70 | 25.60 |
| | Voluma | mg/gr | | | | | | |
| 3 | | 4 | IIA/IIIB | 0.8% | 4.00 | 0.37 | 0.56 | 3.68 |
| | | mg/gr | | | | | | |
| 4 | | 6 | IIA/IIIB | 0.8% | 2.60 | 0.12 | 1.14 | 2.56 |
| | | mg/gr | | | | | | |
| 5 | | 8 | IIA/IIIB | 0.8% | 2.20 | 0.22 | 0.93 | 2.13 |
| | | mg/gr | | | | | | |
| 6 | | 4 | IIA/IIIB | 1.0% | 2.80 | 0.01 | 1.21 | 2.50 |
| | | mg/gr | | | | | | |
| 7 | | 6 | IIA/IIIB | 1.0% | 2.20 | 0.64 | 1.52 | 2.18 |
| | | mg/gr | | | | | | |
| 8 | | 8 | IIA/IIIB | 1.0% | 1.30 | 0.08 | 0.75 | 1.25 |
| | | mg/gr | | | | | | |

Entry 1, 2 represent two commercial dermal fillers with crosslinking degrees of 1% and 6%, with a different linker, which include a fraction of soluble HA higher than 10% (16.4% and 26.7%, respectively).

Entries 3-8 represent exemplary compositions of the invention, exhibiting the effects of (i) crosslinking degrees in a range of 0.8%-1.0% and (ii) of HA concentrations in a range of 4 mg/gr-8 mg/gr, which comply with a soluble HA fraction lower than 10%.

Entries 1, 2 versus Entries 3-8 confirm low sHA content of the exemplary compositions of the invention, as opposed to the commercially available gels. Specifically, the compositions of the invention comprise a very low content of the sHA fraction having a molecular weight lower than 50 kDa, which is associated with a significant pro-inflammatory risk, as disclosed in the literature.

Additionally, the inventors evaluated the sHA content of HA gels of the invention, comprising styrene/tetrazine-based cross linkers (e.g. cross linker derived from the styrene and tetrazine moieties IA and IIIB, respectively). Some of the exemplary compositions comprising styrene/tetrazine-based cross linkers with a crosslinking degree greater than 1% exhibited sHA content within the acceptable range.

### Example 2

### Rheology, Extrusion Forces and HA content in the Soluble Fraction

Exemplary compositions comprising cross-linked polymers and unmodified HA of the invention have been tested for the rheology, extrusion forces and sHA content of each composition obtained according to different procedures (Procedures A-C).

Additionally, the extrusion force for each tested composition has been determined, as described hereinabove. The results of these tests are summarized in Tables 3 and 4.

### Determination of the HA Content in the Soluble Fraction ((Arbazole Assay)

The gels (about 200 mg) were weighted into a 15 mL falcon tube. An excess of 7 mL of phosphate buffered saline (PBS) was added to each tube. The resulting mixtures were homogenized by vortex and incubated of 7 hours at 37° C., while the samples were gently rotated. After incubation, the samples were filtered through 0.45 μm Nylon filter to separate the non-soluble gel from the soluble HA fraction in the filtrates. The filtrate samples were diluted with PBS in order to fit in the assay calibration range and then the HA concentration was determined using a Carbazole Assay (adapted from the Assay described in the Sodium Hyaluronate monograph in the European Pharmacopoeia). The HA concentrations in the filtrate were converted into the corresponding percentage of HA content from the soluble fraction in the initial gel. Experiments were conducted in triplicate.

The inventors found that the mixing of unmodified HA with HA-tetrazine and HA-norbornene allows to produce the expected advantage without damaging the gel homogeneity and therefore with a limited reduction of the mechanical properties.

Procedure A: no unmodified HA added.

Procedure B, according to the invention: HA-tetrazine, HA-norbornene and unmodified HA mixed simultaneously. Alternatively, an aqueous solution of HA-tetrazine or of HA-norbornene has been first mixed with a predetermined amount of unmodified HA and the resulting mixture was subsequently reacted with HA-norbomene or with HA-tetrazine, respectively. The reaction has been performed under conditions described in the Materials and Methods section.

Procedure C: unmodified HA added after crosslinking reaction and homogenized mechanically.

TABLE 3
Rheology and HA content in the soluble fraction (as determined by carbazole assay)

| | | | | | | | | % HA |
|---|---|---|---|---|---|---|---|---|
| | Procedure | | | | | | | in the |
| | or Product | HA | | Crosslinking | Unmodified | G′ | δ | sol. |
| Ent. | name | (mg/gr) | Linker | degree | HA | (Pa) | (°) | fraction |
| 1 | A | 6 | IIA/IIIB | 1.4% | 0 | 107 | 1.0 | 1.3% |
| | | mg/gr | | | | | | |
| 2 | B | 6 | IIA/IIIB | 1.4% | 20% | 69 | 4.7 | 16.7% |
| | | mg/gr | | | | | | |
| 3 | C | 6 | IIA/IIIB | 1.4% | 20% | 79 | 12.9 | 17.5% |
| | | mg/gr | | | | | | |
| 4 | A | 8 | IIA/IIIB | 1.9% | 0 | 284.6 | 1.0 | 0.1% |
| | | mg/gr | | | | | | |
| 5 | B | 8 | IIA/IIIB | 1.9% | 10% | 213.6 | 2.2 | 2.9% |
| | | mg/gr | | | | | | |
| 6 | B | 8 | IIA/IIIB | 1.9% | 20% | 175.0 | 3.2 | 8.7% |
| | | mg/gr | | | | | | |
| 7 | C | 8 | IIA/IIIB | 1.9% | 20% | 138.3 | 14.1 | 20.0% |
| | | mg/gr | | | | | | |
| 8 | A | 8 | IIA/IIIB | about 3% | 0% | 212 | 0.8 | 0.6% |
| | | mg/gr | | | | | | |
| 9 | B | 8 | IIA/IIIB | about 3% | 5% | 173 | 1.2 | 4.0% |
| | | mg/gr | | | | | | |
| 10 | B | 8 | IIA/IIIB | about 3% | 10% | 163 | 2.2 | 7.4% |
| | | mg/gr | | | | | | |
| 11 | B | 8 | IIA/IIIB | about 3% | 15% | 159 | 3.1 | 11.2% |
| | | mg/gr | | | | | | |
| 12 | B | 2 | IIA/IIIB | 2.21% | 10% | 9 | 6.2 | 9.2% |
| | | mg/gr | | | | | | |
| 13 | B | 4 | IIA/IIIB | 2.21% | 10% | 48 | 2.1 | 4.9% |
| | | mg/gr | | | | | | |
| 14 | B | 8 | IIA/IIIB | 2.21% | 10% | 221 | 1.6 | 2.8% |
| | | mg/gr | | | | | | |
| 15 | B | 10 | IIA/IIIB | 2.21% | 10% | 334 | 1.7 | 2.7% |
| | | mg/gr | | | | | | |
| 16 | B | 4 | IIA/IIIB | 2.21% | 30% | 33 | 4.0 | 21.5% |
| | | mg/gr | | | | | | |

Entries 2-3 versus Entry 1, Entries 5-7 versus Entry 4 and Entries 9-10 versus Entry 8 show that the addition of unmodified HA has the expected effect to decrease rheological properties as shown by a decrease in G′ and increase in phase angle δ.

The G′ values of the gels prepared with Procedure B and C are comparable (about 10% to 20% difference), as shown by Entry 2 vs Entry 3 and Entry 6 vs Entry 7.

The phase angle δ of gels prepared with Procedure B are significantly lower than those of gels prepared with Procedure C (175% to 340%), as shown by Entry 2 vs Entry 3 and Entry 6 vs Entry 7. These data indicate that stronger gels, more resistant and of higher mechanical quality are produced from procedure B than from Procedure C. Therefore, Procedure B allows to better preserve the overall viscoelastic properties of the gel, by preserving lower phase angle δ values.

Entries 5 and 6, as well as Entries 9-11, show the dose-effect of the content in unmodified HA in the compositions, when prepared according to Procedure B. There is a gradual effect on the decrease in G′ and increase of phase angle δ from 10% to 20% of unmodified HA in the composition of Entries 5-6 and from 5% to 15% of unmodified HA in the composition of Entries 9-11.

Entries 2 and 3 show that the measured amounts of HA in the soluble fraction of the prepared gels are similar from both Procedures B and C (Entries 2 vs 3, 16.7% versus 17.5%), confirming that the added unmodified HA is not interconnected to the cross-linked matrix when the gel are prepared at a crosslinking degree of 1.4% and a HA concentration of 6 mg/gr.

Entries 5 and 6 and Entries 9-11 show that at a higher crosslinking degree of 1.9% or about 3% and a higher HA concentration of 8 mg/gr, the unmodified HA is not fully recovered as soluble HA fraction from gels prepared according to Procedure B (% HA in the soluble fraction of 2.9% and 8.7%, for a composition containing respectively 10% and 20% of unmodified HA in Entries 5-6 respectively). In such cases, it is suspected that the denser cross-linked network hinders the release of the unmodified HA in water.

Entries 12-15 show the effect of HA concentration on the formulation prepared according to Procedure B with a constant crosslinking degree of 2.21% and a constant amount of unmodified HA added to the composition. Increase in the HA concentration in the composition from 2 mg/gr (Entry 12) to 10 mg/gr (Entry 15) have the effect to increase the G′ and decrease the phase angle δ, reflecting the hardening of the gel. It is also observed that the %HA in the soluble fraction, which is close to 10% for the least concentrated formulation (Entry 12), i.e. the amount added to the formulation, gradually decreases down to 2.7% for the highest concentrated composition (Entry 15). This observation is supporting the formerly mentioned hypothesis that a denser HA network hinders the release of the unmodified HA in water.

Entry 16 shows that adding more unmodified HA to the composition (30%) allows to create a very soft gel (low G′) although highly elastic (low phase angle δ), probably very well suited for shallower dermal injections.

TABLE 4
Extrusion force values

| | | | | | | Extrusion | |
|---|---|---|---|---|---|---|---|
| | Procedure | | | | | force | Extrusion |
| | or Product | | | Crosslinking | Unmodified | average | force max |
| Ent. | name | HA | Linker | degree | HA | (N) | (N) |
| 1 | A | 6 | IIA/IIIB | 1.4% | 0 | 21.5 | 22.8 |
| | | mg/gr | | | | | |
| 2 | B | 6 | IIA/IIIB | 1.4% | 20% | 6.4 | 7.5 |
| | | mg/gr | | | | | |
| 3 | C | 6 | IIA/IIIB | 1.4% | 20% | 4.1 | 12.3 |
| | | mg/gr | | | | | |
| 4 | A | 8 | IIA/IIIB | 1.9% | 0 | 26.5 | 28.2 |
| | | mg/gr | | | | | |
| 5 | B | 8 | IIA/IIIB | 1.9% | 10% | 9.6 | 11.0 |
| | | mg/gr | | | | | |
| 6 | B | 8 | IIA/IIIB | 1.9% | 20% | 5.5 | 6.3 |
| | | mg/gr | | | | | |
| 7 | C | 8 | IIA/IIIB | 1.9% | 20% | 2.6 | 5.5 |
| | | mg/gr | | | | | |
| 8 | A | 8 | IIA/IIIB | 2.74% | 0% | 26.1 | 27.5 |
| | | mg/gr | | | | | |
| 9 | B | 8 | IIA/IIIB | 2.74% | 5% | 17.0 | 18.0 |
| | | mg/gr | | | | | |
| 10 | B | 8 | IIA/IIIB | 2.74% | 10% | 12.0 | 13.1 |
| | | mg/gr | | | | | |
| 11 | B | 8 | IIA/IIIB | 2.74% | 15% | 8.6 | 9.6 |
| | | mg/gr | | | | | |
| 12 | B | 2 | IIA/IIIB | 2.21% | 10% | 3.6 | 4.7 |
| | | mg/gr | | | | | |
| 13 | B | 4 | IIA/IIIB | 2.21% | 10% | 7.6 | 8.0 |
| | | mg/gr | | | | | |
| 14 | B | 8 | IIA/IIIB | 2.21% | 10% | 12.4 | 13.2 |
| | | mg/gr | | | | | |
| 15 | B | 10 | IIA/IIIB | 2.21% | 10% | 16.5 | 17.6 |
| | | mg/gr | | | | | |
| 16 | B | 4 | IIA/IIIB | 2.21% | 30% | 3.9 | 4.3 |
| | | mg/gr | | | | | |

Entries 2-3 versus Entry 1 and Entries 5-7 versus Entry 4 and Entries 9-11 versus Entry 8, show that the addition of unmodified HA has the effect to decrease the average gel's extrusion force, allowing an easier injection to patients.

The average extrusion forces of the gels prepared with Procedure B and C are comparable, as shown by Entry 2 vs Entry 3 and Entry 6 vs Entry 7.

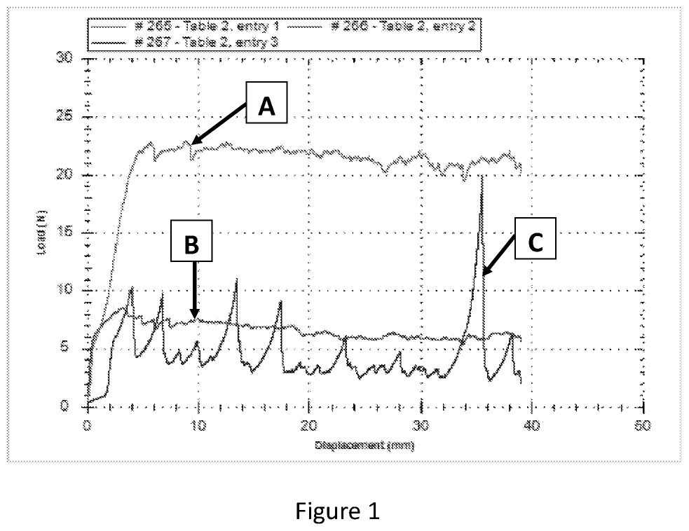

Procedure B affords a more homogeneous gel than Procedure C, as shown by the smaller difference between the maximum and average extrusion forces: respectively 1.1 N and 8.2 N for Entry 2 versus Entry 3 and respectively 0.8 N and 2.3 N for Entry 6 versus Entry 7. Better gel homogeneity and smoother extrusion profile of gels prepared by Procedure B versus Procedure C are also visually observed in FIG. 1.

Noteworthy, the homogeneity and linearity of the extrusion profile could be improved for gels prepared according to Procedure C, by stronger gel sieving, mixing or other mechanical shearing process. However, the gel mechanical quality would simultaneously decrease (decrease in G′ and increase in phase angle δ) which is not advantageous for dermal fillers.

Entries 5 and 6, Entries 9-11 and Entries 13 and 16 show the dose-effect of the content in unmodified HA in the compositions, when prepared according to Procedure B. There is a gradual effect on the decrease in average extrusion force from 10% to 20% (Entries 5-6) or from 5% to 15% (Entries 9-11) or from 10% to 30% (Entries 13 and 16) of unmodified HA in the composition, while the gel homogeneity is good in all cases, as shown by the difference between maximum and average extrusion forces (respectively 1.4 N, 0.8 N for Entry 5 and 6 and 1.0 N, 1.1 N, 1.0 N for Entries 9-11 and 0.4 N for Entries 13 and 16).

Entries 12-15 show the effect of HA concentration on the formulation prepared according to Procedure B with a constant crosslinking degree of 2.21% and a constant amount of unmodified HA added to the composition. Increase in the HA concentration in the composition from 2 mg/gr (Entry 12) to 10 mg/gr (Entry 15) leads to a gradual increase of the average extrusion force, while the gel homogeneity is good in all cases, as shown by the difference between maximum and average extrusion forces (respectively 1.1 N, 0.4 N, 0.8 N and 1.1 for Entries 12-15).

### Example 3

### HPLC Determination of HA Content in the Soluble Fraction

The content of HA and sHA (determined as described hereinabove) in the soluble fraction of the compositions of the Example 2 was determined by HPLC-MALS-RI (Table 5).

TABLE 5
HA content in the soluble fraction (as determined by HPLC)

| | | | | | | | % sHA | % sHA | % sHA |
|---|---|---|---|---|---|---|---|---|---|
| | | | | | | % HA in | with | with | with |
| | | | | Crosslinking | Unmodified | soluble | MW <50 | MW <100 | MW <250 |
| Ent. | Procedure | HA | Linker | degree | HA | fraction | kDa | kDa | kDa |
| 1 | A | 6 | IIA/IIIB | 1.4% | 0 | 1.01 | 0.32 | 0.75 | 0.94 |
| | | mg/gr | | | | | | | |
| 2 | B | 6 | IIA/IIIB | 1.4% | 20% | 9.59 | 0 | 0 | 0 |
| | | mg/gr | | | | | | | |
| 3 | C | 6 | IIA/IIIB | 1.4% | 20% | 11.09 | 0 | 0 | 0 |
| | | mg/gr | | | | | | | |

It can be observed that the HA content in the soluble fraction and the contents of HA fragments of low molecular weights are inferior to 1%, in the gel prepared in the absence of unmodified HA (Table 5, Entry 1).

The amount of HA in the soluble fraction is similar in the comparable gels prepared by methods B and C (Table 5, Entries 2-3, 9.89% versus 11.09%), as determined by HPLC.

Fragments of HA with low molecular weights, and higher pro-inflammatory risk, were not detected in the two comparable gels prepared by methods B and C (Entries 2-3).

The average molecular weight of HA in the soluble fraction is similar in the comparable gels prepared by methods B and C (612.3 kDa versus 711.1 kDa), as determined by HPLC.

### Example 4

### Dissolution Experiments in Water

About 200 mg of gel was extruded from the syringe and 8 mL of water colored with Toluidine blue (0.0025%) was added. After 3 gentle mixtures, the gel was left to stand in the colored water and evolution was visually observed.

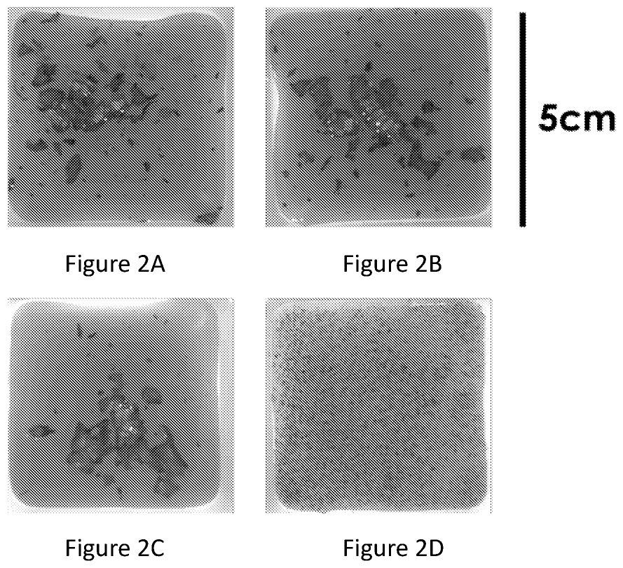

FIGS. 2A-D show the dissolution test result after 30 minutes for the gels obtained according to Entries 4-7 of Table 3 of the Example 2.

Similar results were obtained with the gel from Procedure A of the Example 2, prepared without unmodified HA (Entry 4, Table1) (FIG. 2A), and the two gels from Procedure B of the Example 2 (Entry 5,6, Table 3 of the Example 2) (FIGS. 2B-C), with some large-sized fragments (>5 mm) resulting from the random fragmentation of the initially homogeneous gel. On the contrary, the gel from Procedure C of the Example 2 (Entry 7, Table 3) was dissolved into small particles (<2 mm) (FIG. 2D).

These results show good homogeneity of the cross-linked gel when HA-tetrazine, HA-norbornene and unmodified HA are mixed simultaneously, according to Procedure B. On the other hand, the use of Procedure C leads to massive gel's fragmentation into small particles, with expected negative impact on the final cross-linked gel mechanical quality.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising a polymer comprising a first hyaluronic acid (HA) chain or a derivative thereof and a second HA chain or a derivative thereof, wherein said first HA chain and said second HA chain are crosslinked via one or more linkers, wherein said one or more linkers comprise an unsaturated moiety or a derivative thereof bound to a tetrazine moiety or a derivative thereof, wherein said polymer is characterized by a crosslinking degree of between 0.6 and 4%, and wherein a weight ratio between water soluble HA (sHA) and said polymer within said composition is less than 10%.

2. The composition of claim 1, wherein said one or more linkers is represented by 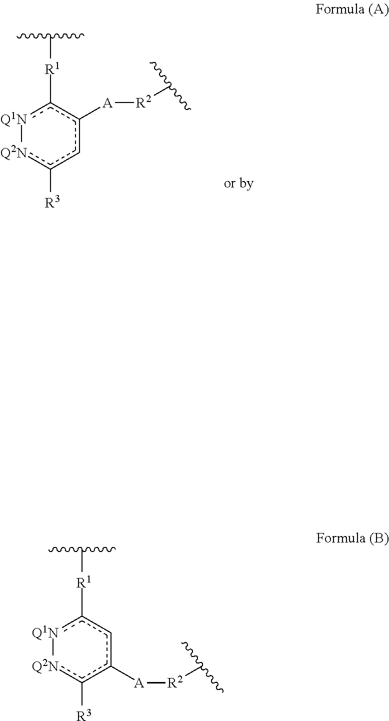
or a combination thereof; wherein: [symbol]
represents a single or a double bond;
R¹, R², or both, are selected from the group consisting of: a bond, alkyl, alkenyl, cycloalkyl, aryl, heteroaryl, heteroalicyclic, C(O)—NH-alkyl-NH, and alkyl-NZ wherein Z is a bond, aryl, or heteroaryl;
A is selected form the group consisting of: a bond, alkyl, and aryl;
Q¹, Q² or both represent hydrogen, or are absent;
and wherein R³ is selected from the group consisting of: hydrogen, alkyl, aryl, or heteroaryl substituted or non-substituted.

3. The composition of claim 1, wherein said unsaturated moiety comprises a styrene moiety or a derivative thereof; and wherein said styrene moiety is covalently bound to said first HA chain or to said second HA chain; and wherein said styrene moiety or a derivative thereof is represented by formulae IA-D:

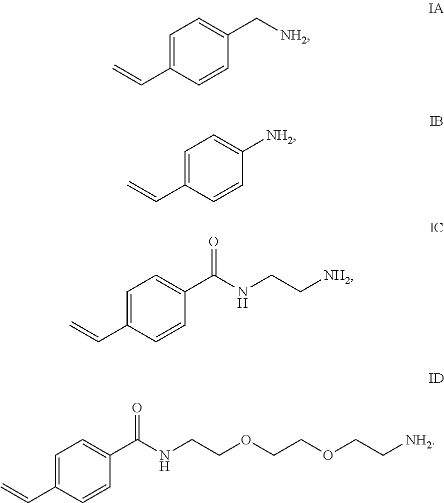

4. The composition of claim 1, wherein said one or more linkers is represented by 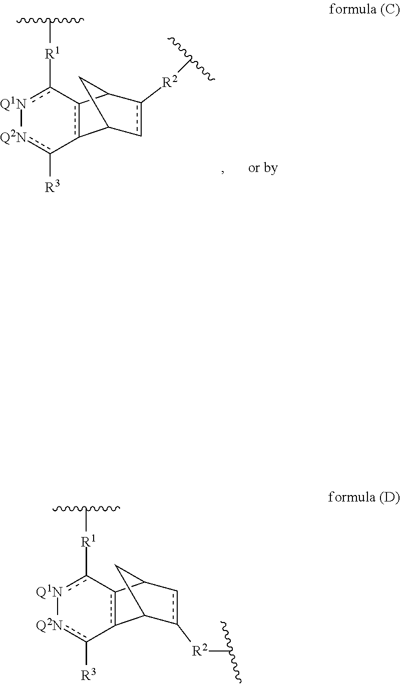
or a combination thereof; wherein: [symbol]
represents a single or a double bond;
R¹, R², or both, are selected from the group consisting of: a bond, alkyl, alkenyl, cycloalkyl, aryl, heteroaryl, heterocyclyl, C(O)—NH-alkyl-NH , and alkyl-NZ wherein Z is a bond, aryl, or heteroaryl;
Q¹, Q² or both represent hydrogen, or are absent; and wherein
R³ is selected from the group consisting of: hydrogen, alkyl, aryl, or heteroaryl substituted or non-substituted.

5. The composition of claim 4, wherein said one or more linkers is derived from:
(a) a norbornene moiety represented by any of formulae IIA-G: 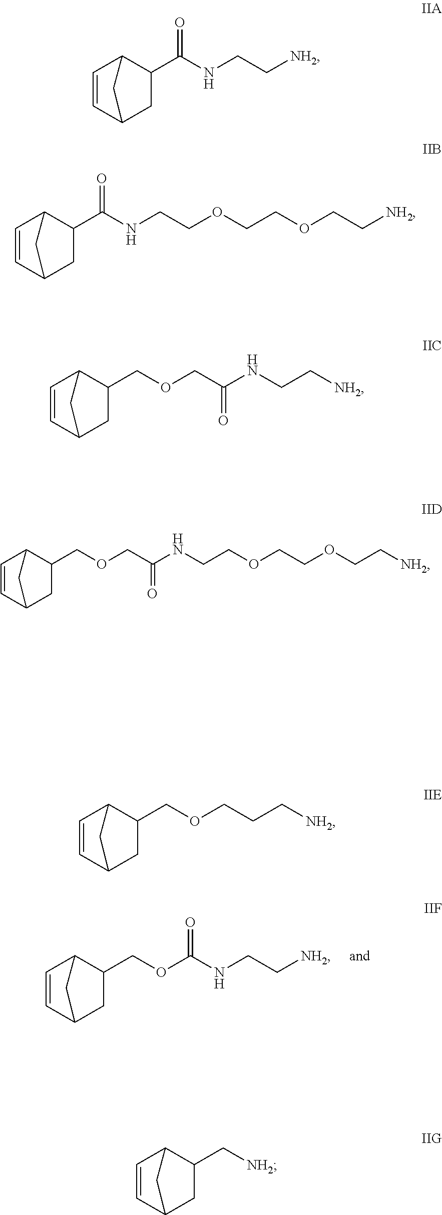
and from (b) a tetrazine moiety represented by any of formulae IIIA-F: 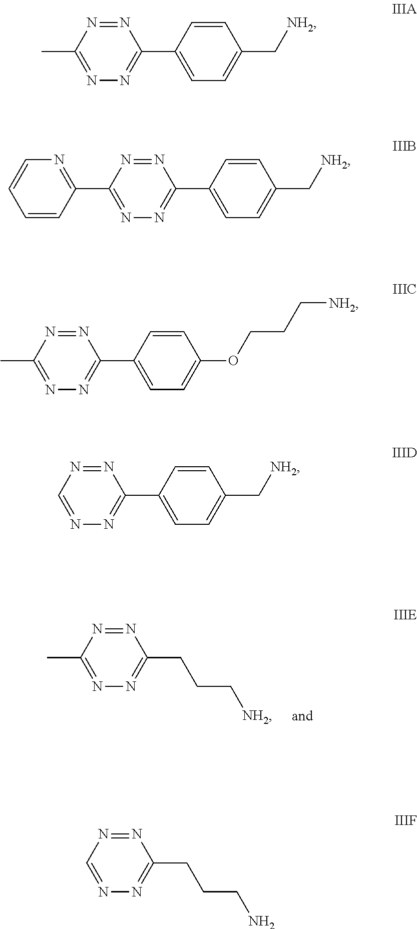 [SMILES: Cc1nnc(-c2ccc(CN)cc2)nn1 ; NCc1ccc(-c2nnc(-c3ccccn3)nn2)cc1 ; Cc1nnc(-c2ccc(OCCCN)cc2)nn1 ; NCc1ccc(-c2nncnn2)cc1 ; Cc1nnc(CCCN)nn1 ; NCCCc1nncnn1]
wherein each of said one or more linkers comprises said norbornene moiety covalently bound to said tetrazine moiety wherein any one of said norbornene moiety and said tetrazine moiety is covalently bound to (i) said first HA chain or (ii) to said second HA chain via an amide bond.

6. (canceled)

7. (canceled)

8. (canceled)

9. The composition of claim 1, wherein the crosslinking degree is between 0.7% and 3%; and wherein at least 70% by weight of said first HA chain and of said second HA chain within said composition are cross-linked wherein said first HA chain and said second HA chain have an average molecular weight (MW) of 500,000 to 4,000,000 Daltons (Da), and wherein said polymer is characterized by water solubility of at most 0.1 g/L.

10. (canceled)

11. The composition of claim 1, further comprising an aqueous solution, and wherein a concentration of said polymer within said composition is between 1 and 10 mg/ml; wherein said sHA is characterized by an average molecular weight (MW) of less than 250 kDa, and by water solubility of at least 0.1 g/L.

12. (canceled)

13. The composition of claim 11, further comprising an unmodified HA chain characterized by an average molecular weight (MW) between 300 kiloDaltons (kDa) and 4,000 kDa, and wherein said composition is characterized by a phase angle (δ) up to 10°; wherein said composition is characterized by being substantially devoid of particulate matter; wherein said composition comprises between 5% and 30% weight per weight (w/w) of said unmodified HA chain; and wherein said composition comprises between 5% and 30% weight per weight (w/w) of said unmodified HA chain; and wherein said unmodified HA is characterized by water solubility of at least 1 g/L.

14. (canceled)

15. (canceled)

16. The composition of claim 11, wherein said composition is characterized by at least one of: an average extrusion force between 5 newton (N) and 30 N; a maximum extrusion force between 5 N and 30 N; extrusion profile devoid of peaks higher than 2 N; and an elastic modulus (G′) between 20 Pa and 400 Pa; wherein said composition is characterized by an extrusion force of less than 30 N; and wherein the cross-linking a degree of said polymer is predetermined by a molar ratio between (i) any one of: said norbornene moiety, said tetrazine moiety, or said styrene moiety; and (ii) said first HA chain, or said second HA chain, as determined by ¹H NMR.

17. A method for filling or volumizing a tissue in a subject in need thereof, comprising administering the composition of claim 1.

18. The method of claim 17, wherein said tissue is selected from the group consisting of: skin, gingival, cartilage and ophthalmic tissue, muscles, and subcutaneous tissues; optionally wherein said administering comprises subcutaneous administration.

19.-25. (canceled)

26. A composition comprising:
(a) a polymer comprising a first hyaluronic acid (HA) chain or a derivative thereof and a second HA chain or a derivative thereof, wherein:
(i) said first HA chain and said second HA chain are cross-linked via one or more linkers;
(ii) said one or more linkers comprise an unsaturated moiety or a derivative thereof and a tetrazine moiety or a derivative thereof;
(iii) said cross-linked is characterized by a crosslinking degree between 0.6% and 4%; and
(b) an unmodified HA chain characterized by an average molecular weight (MW) between 300 kiloDaltons (kDa) and 4,000 kDa, and wherein said composition is characterized by a phase angle (δ) up to 10°.

27. The composition of claim 26, wherein said composition comprises between 5% and 30% weight per weight (w/w) of said unmodified HA chain; and wherein said composition is characterized by being substantially devoid of particulate matter; and wherein said composition is characterized by at least one of: an average extrusion force between 5 newton (N) and 30 N; a maximum extrusion force between 5 N and 30 N; extrusion profile devoid of peaks higher than 2 N; and an elastic modulus (G′) between 20 Pa and 400 Pa.

28. (canceled)

29. The composition of claim 26, wherein a concentration of said polymer within said composition is between 1 mg/ml and 25 mg/ml; and
wherein said first HA chain and said second HA chain is characterized by an average MW between 500 kDa and 4,000 kDa.

30. The composition of claim 26, wherein said one or more linkers is represented by 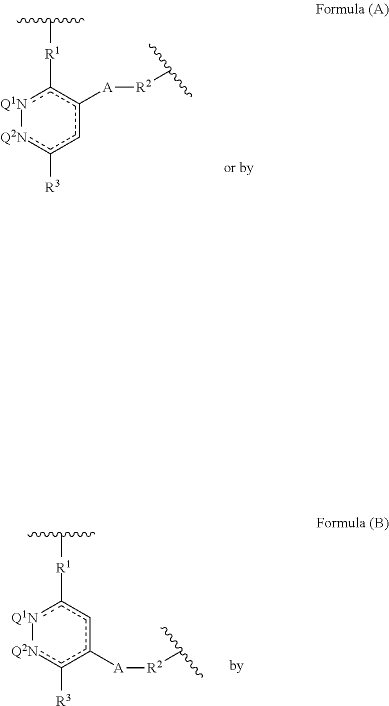 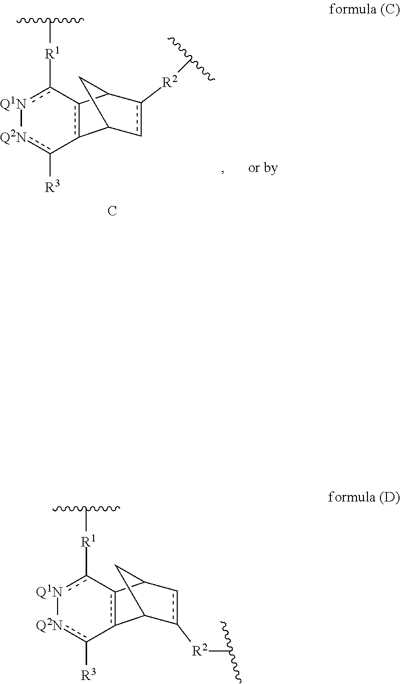
or a combination thereof, wherein: [symbol]
represents a single or a double bond;
R¹, R², or both, are selected from the group consisting of: a bond, alkyl, alkenyi, cycloalkyl, aryl, heteroaryl, heteroalicyclic, C(O)—NH-alkyl-NH, and alkyl-NZ wherein Z is a bond, aryl, or heteroaryl;
A is selected form the group consisting of: a bond, alkyl, and aryl;
Q¹, Q² or both represent hydrogen, or are absent;
and wherein R³ is selected from the group consisting of: hydrogen, alkyl, aryl, or heteroaryl substituted or non-substituted.

31. The composition of claim 30, wherein said unsaturated moiety comprises a styrene moiety or a derivative thereof and wherein said styrene moiety is covalently bound to said first HA chain or to said second HA chain. wherein said styrene moiety or a derivative thereof is represented by formulae IA-D:

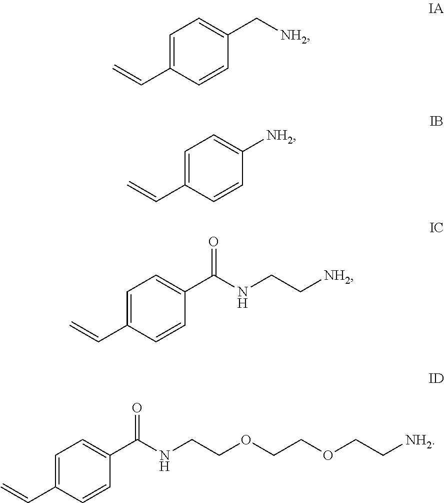

32. The composition of claim 26, wherein each of said one or more linkers comprises said norbornene moiety covalently bound to said tetrazine moiety; wherein any one of said norbornene moiety and said tetrazine moiety is covalently bound to (i) said first HA chain or (ii) to said second HA chain.

33. (canceled)

34. The composition of claim 32, wherein said one or more linkers is derived from:
(a) a norbornene moiety represented by any of formula IIA-G: 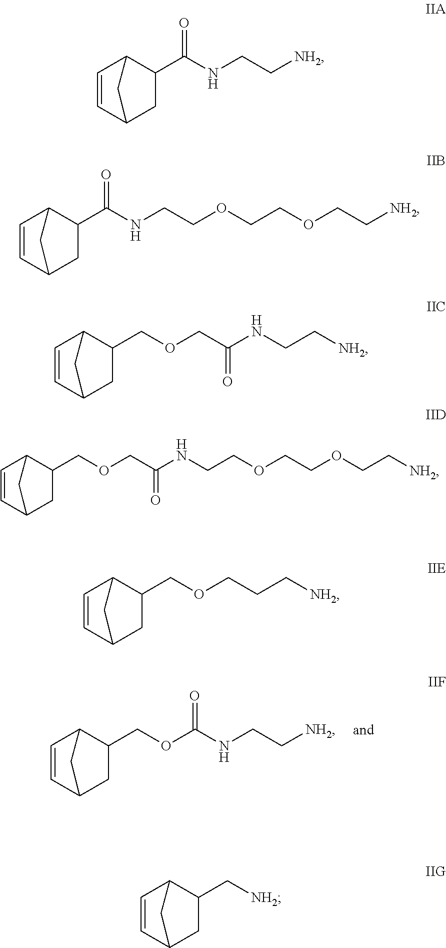
and from (b) a tetrazine moiety represented by any of formulae IIIA-F:

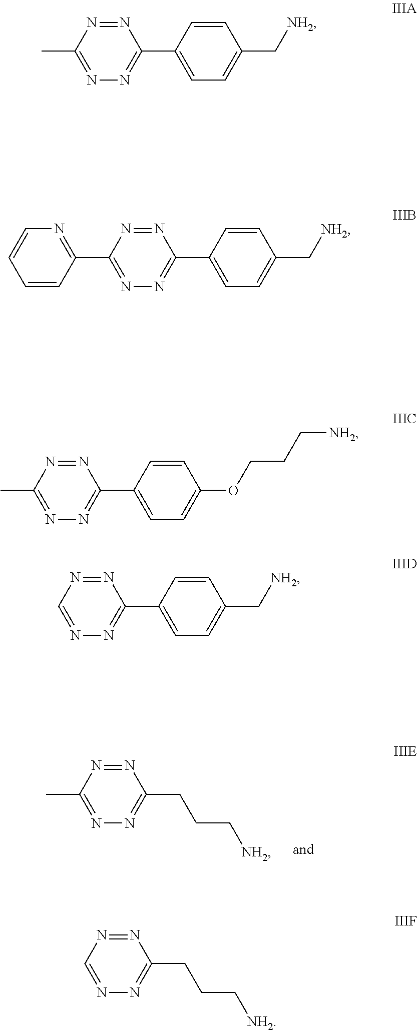 [SMILES: Cc1nnc(-c2ccc(CN)cc2)nn1 ; NCc1ccc(-c2nnc(-c3ccccn3)nn2)cc1 ; Cc1nnc(-c2ccc(OCCCN)cc2)nn1 ; NCc1ccc(-c2nncnn2)cc1 ; Cc1nnc(CCCN)nn1 ; NCCCc1nncnn1]

35.-44. (canceled)
